(19) United States
(12) Patent Application Publication
(10) Pub. No.: US 2025/0164874 A1
(43) Pub. Date: May 22, 2025

(54) LITHOGRAPHY COMPOSITIONS AND METHODS FOR FORMING RESIST PATTERNS AND/OR MAKING SEMICONDUCTOR DEVICES

(71) Applicant: The Research Foundation for the State University of New York, Albany, NY (US)

(72) Inventors: Robert Brainard, Albany, NY (US); Munsaf Ali, Albany, NY (US)

(21) Appl. No.: 18/839,581

(22) PCT Filed: Feb. 20, 2023
(86) PCT No.: PCT/US23/13410

§ 371 Date: Aug. 19, 2024

Related U.S. Application Data
(60) Provisional application No. 63/311,697, filed on Feb. 18, 2022.

(51) Int. Cl. G03F 7/004; G03F 7/16

(52) U.S. Cl. CPC G03F 7/0042; G03F 7/162

(57) ABSTRACT

The present disclosure relates to compounds and use thereof as film or lithographic compositions such as EUV photoresist films. More particularly, embodiments of the disclosure provide lithography compositions and methods of depositing radiation sensitive films, which can be used for patterning applications with UV light, EUV light or electron-beam radiation to form high resolution patterns with low line width roughness. In embodiments, novel ligands are provided for forming radiation sensitive film compositions.

### CROSS-REFERENCE TO RELATED APPLICATIONS

This application claims the benefit of the filing date of U.S. Provisional Application No. 63/311,697, which was filed Feb. 18, 2022. The content of this earlier filed application is hereby incorporated by reference herein in their entirety.

### FIELD OF THE INVENTION

The present disclosure is generally in the fields of organic chemistry and electronic device manufacturing. More particularly, embodiments of the disclosure provide lithography compositions and methods of depositing films such as radiation sensitive films, which can be used for patterning applications with UV light, EUV light or electron-beam radiation to form high resolution patterns with low line width roughness.

### BACKGROUND

In forming electronic devices such as semiconductor-based structures, materials are patterned to integrate a predetermined structure. Structures are typically formed through sequential deposition and etching steps through which a pattern is formed of the various materials. In this way, many devices such as transistors can be formed in a high-density area.

Organic compositions can be used as patterned resists such as radiation patterned resists, so that a pattern alters the chemical structure of the organic compositions corresponding with the pattern. For example, processes for the patterning of semiconductor wafers can include lithographic transfer of a desired image from a thin film of organic radiation-sensitive material. The patterning of the resist generally involves several process sequences including exposing the resist to an energy source, such as through a mask, to record a latent image and then developing and removing selected regions of the resist. For a positive-tone resist, the exposed regions are altered to make such regions selectively removable, while for a negative-tone resist, the unexposed regions are selectively removable.

Generally, the pattern is formed using radiation to alter a portion of the resist while the other portions of the resist act as a protective layer such as an etch-resistant layer. The substrate can be selectively etched through holes in the remaining areas of the protective resist layer. Alternatively, materials can be deposited into the exposed regions of the underlying substrate through the developed holes in the remaining areas of the protective resist layer. Ultimately, the protective resist layer is removed. Generally, the process can be repeated to form additional layers of patterned material. Additional processing sequences can be used, such as the deposition of conductive materials or implantation of dopants. In the fields of micro- and nanofabrication, feature sizes in integrated circuits have become very small to achieve high-integration densities and improve circuit function.

Prior art-of-interest includes U.S. Pat. No. 10,228,618 entitled Organotin oxide hydroxide patterning compositions, precursors, and patterning (herein incorporated entirely by reference), however, the methods do not provide high resolution lithography patterning coatings based on the chemistry, compositions, and/or methods of the present disclosure.

Prior art-of-interest also includes U.S. Pat. No. 11,156,920 entitled Lithography composition, a method for forming resist patterns and a method for making semiconductor devices (herein incorporated entirely by reference), however, the disclosure does not provide the compositions of the present disclosure.

As microelectronic device sizes become smaller, challenges exist with current resist films. New films, such as photolithographic films, are needed for a variety of applications in microelectronic devices. Accordingly, there is a continuing need in the art for new lithography compositions, methods for forming resist patterns using one or more lithography compositions, and semiconductor device manufacturing methods using film and/or lithography compositions.

### SUMMARY

In embodiments, the present disclosure provides new film compositions, lithography compositions, methods for forming resist patterns using a film or lithography composition, and semiconductor device manufacturing methods using the film or lithography compositions in a method of the present disclosure.

In embodiments, the present disclosure provides a mononuclear lithographic composition, represented by the below formula, RₐM(O₂CR′)₂, wherein in M is tellurium (Te), antimony (Sb), tin (Sn), or bismuth (Bi), wherein a is 2-3, and wherein at least one of R and R′, include an alkyne functional group moiety.

In embodiments, the present disclosure includes a multinuclear lithographic composition, represented by the below formula, [MₐN_{b}O_{c}H_{d}BₑR_{f}X_{g}(WCOR′)ₕ(OR″)ᵢ(WCOCOY)ⱼ(COY)ₖ]l wherein: the composition contains at least one of R, R′ and R″, and at least one of R, R′ and R″ includes an alkyne functional group moiety: R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, -o-C₆H₄OCH₃, -m-C₆H₄OCH₃, -p-C₆H₄OCH₃, —C₆H₄CH₂CH₃, -o-CH₂C₆H₄OCH₃, -m-CH₂C₆H₄OCH₃, -p-CH₂C₆H₄OCH₃, —C₆H₁₁, —C≡CH, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH, —C≡CR′″, —CH₂C₆H₄C≡CR′″, —(CO)C₆H₄C≡CR′″, or —C₆H₄C≡CR′″; R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, -o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH, —C≡CR′″, —C₆H₄C≡CR′″, or —CH₂C₆H₄C≡CR′″; R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, or —C₆H₄C(CH₃)═CH₂, —C₆H₄C≡CH, —CH₂C₆H₄C≡CH, —C₆H₄C≡CR′″, or —CH₂C₆H₄C≡CR′″;
R′″, when present, is independently an aromatic or aliphatic hydrocarbon selected from: —CH₃, —CH₂CH₃, or —C₆H₅;
M is each independently an element selected from the group of tellurium (Te), antimony (Sb), tin (Sn), iodine, (I), and bismuth (Bi);
N, when present, is each independently an element selected from the group consisting of indium (In), tin (Sn), antimony (Sb), tellurium (Te), bismuth (Bi) and iodine (I); H, when present, is each independently hydrogen; O is each independently oxygen;
B, when present, is each independently selected from W, C₂O₄, SO₄, PO₄, (CH₂)₂C₆H₂(CH₂)₂, —C≡C—, —CH₂C≡C—, —CH₂C≡CCH₂—, or —CH₂C₆H₄CH₂—, —O—O—, —O₂CCH₂CH₂CO₂—, —O₂CCH═CHCO₂—, —O₂CC≡CCO₂—, —(OCO)₂NCH₂CH₂N(COO)₂—,
X, when present, is each independently selected from F, Cl, Br, CN, I, or C₂O₄, an alkyne functional group moiety, —O₂CC≡CH, —O₂CC₆H₄C≡CH, or —O₂CCH₂C₆H₄C≡CH;
W, when present, is each independently an element or a compound selected from the group consisting of —CH₂, NR′, S, and O;
Y when present, is each independently an element or compound selected from the group consisting of —WR′, and —R′, wherein a=1-8; b=0-5; c=0-20; d=0-20; e=0-10; f=0-20; g=0-20; h=0-5; i=0-5; j=0-5; k=0-6; and l=charge on an ion or a complex selected from: −4, −3, −2, −1, 0, +1, +2, +3, +4.

In embodiments, the present disclosure relates to a method for forming a resist pattern using a film or lithography composition, including: contacting a substrate with a film or lithography composition of the present disclosure to form a thin radiation sensitive film of the film or lithography composition atop the substrate.

In embodiments, the present disclosure relates to a method for forming a semiconductor device using one or more film compositions or lithography compositions of the present disclosure in a method of the present disclosure such as a photolithography method. In embodiments, a method for forming a semiconductor device includes contacting a substrate with a film or lithography composition of the present disclosure to form a thin radiation sensitive film of the film or lithography composition atop the substrate.

In embodiments, the present disclosure relates to a method for forming an electronic device using the lithography compositions in a photolithography method of the present disclosure. In embodiments, a method for forming an electronic device includes contacting a substrate with a lithography composition of the present disclosure to form a thin radiation sensitive film of the lithography composition atop the substrate.

In embodiments, the present disclosure relates to a substrate and a radiation sensitive coating including a composition of the present disclosure.

In embodiments, the present disclosure relates to a substrate including an inorganic semiconductor layer and a radiation sensitive coating material atop a surface of the inorganic semiconductor layer, wherein the radiation sensitive coating includes a lithographic composition of the present disclosure. In some embodiments, the radiation coating material or film of the present disclosure can be patterned with EUV light at a wavelength of 13.5 nm in a pattern of 16-nm lines, with a line width roughness of no more than about 4 nm. In embodiments, the radiation sensitive coating material can include metal, such as Te, and can include at least 4 weight percent metal and in other embodiments at least about 20 weight percent metal, and in other embodiments at least 51 weight percent metal, or, in embodiments a weight percent of 4-51% based on the total weight of the coating material. In embodiments, the radiation sensitive coating is characterized as amorphous.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIGS. 1A-1C depict three mononuclear photoresists.

FIGS. 2A and 2B depict olefin-containing ligands. FIG. 2A depicts R Ligands and FIG. 2B depicts carboxylate ligands.

FIGS. 3A, 3B and 3C depict data of the present disclosure. FIG. 3A depicts an aerial image of dense lines and spaces calculated using EUV light. FIG. 3B high-aspect ratio resist profiles generated using e-beam lithography. FIG. 3C depicts ideal, non-linear contrast curve for a negative-tone resist.

FIG. 4 depicts negative-tone contrast curves for one tellurium and three antimony olefin-containing resists.

FIG. 5A depicts an alkene that polymerizes in the presence of alkyne. FIG. 5B depicts no polymer is produced from the reaction of diphenylacetylene (DPA) under free-radical conditions at high temperature.

FIGS. 6A-6G depict ligand embodiments in mononuclear metal-containing EUV resists and their use in multinuclear complexes of type E (M=Te, Sb, Sn or Bi). FIG. 6H depicts a general formula for multinuclear complexes of the present disclosure.

FIG. 7 depicts contrast curves comparing mononuclear complexes containing alkynes and alkenes. In all cases, the complexes containing alkyne ligands show superior contrast curves (vs. alkene ligands) with either cleaner toes, higher contrast or both.

FIG. 8 depicts mononuclear complexes of tellurium that have been evaluated using EUV lithography shown in bold with the alpha numeric labels. The areas marked “This Embodiment” define compound embodiments of the present disclosure using the new alkynyl ligands that are synthesize and evaluated lithographically herein.

FIG. 9 depicts mononuclear complexes of tin that have been evaluated using EUV lithography shown in bold with the alpha numeric labels. The areas marked “This Embodiment” define compound embodiments of the present disclosure suitable for use herein.

FIG. 10 depicts mononuclear complexes of antimony that have been evaluated using EUV lithography in bold with the alpha numeric labels. The areas marked “This Embodiment” define compounds of the present disclosure using the new alkynyl ligands disclosed herein.

FIG. 11 depicts mononuclear complexes of bismuth that have been evaluated using EUV lithography by our group shown in bold with the alpha numeric labels. The areas marked “This Embodiment” define compounds using the new alkynyl ligands suitable for use in accordance with the present disclosure.

FIG. 12 depicts multinuclear target compounds suitable for use in accordance with the present disclosure.

FIG. 13 depicts a flow diagram illustrating a process 100 for forming an electronic device such as a semiconductor device using the lithography compositions in a photolithography method of the present disclosure.

FIGS. 14A-14C show a schematic diagram of an electronic device such as a semiconductor device, according to embodiments of the present disclosure.

FIG. 15 depicts scanning electron micrographs showing dense-line patterns of olefin resist MA-258 after exposure at PSI using interference lithography and development using 2-heptanone for 30 seconds. In embodiments, such an olefin resist clearly resolves 50-nm dense lines, but struggles with line-collapse and resolution of 40- and 30-nm lines.

FIG. 16 depicts scanning electron micrographs showing dense-line patterns of alkyne-containing resist MA-257 after exposure at PSI using interference lithography and development using 2-heptanone for 30 seconds. In embodiments, such an alkyne-containing resist clearly resolves 50-, 40- and 30-nm dense lines with good sensitivity and good line-edge-roughness. It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

FIG. 17 depicts contrast curve for MN2-133 exhibiting a clean toe.

FIG. 18 depicts contrast curve for the olefin-containing resist JP-20 in comparison to FIG. 19 which shows an alkyne-containing resist, MN2-113. The alkyne-containing resist shows better contrast than the alkene-containing resist.

FIG. 20 depicts scanning electron micrographs showing dense-line patterns of alkyne-containing resist MA-257 after exposure at PSI using interference lithography and development using toluene for 30 seconds. In embodiments, such an alkyne-containing resist clearly resolves 50-, 40- and 30-nm dense lines with good sensitivity and good line-edge-roughness.

FIG. 21 depicts contrast curve for multinuclear resist MA-306 exhibiting a clean toe and a very high contrast. MA-306 contains a 3-carbon bridging group.

FIG. 22 depicts contrast curve for multinuclear resist MA-311. MA-311 contains a peroxide bridging group.

FIG. 23 depicts three multinuclear resists made using tin and antimony and using 2-carbon, 3-carbon and peroxide bridging groups.

### DETAILED DESCRIPTION

The presently disclosed subject matter will now be described more fully and representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Throughout the specification and claims, a given chemical formula or name shall encompass all optical and stereoisomers, as well as racemic mixtures where such isomers and mixtures exist.

In embodiments, the present disclosure provides new lithography compositions, methods for forming of resist patterns using a lithography composition, and semiconductor device manufacturing methods using the lithography compositions in a photolithography method of the present disclosure.

In embodiments, the present disclosure provides a mononuclear lithographic composition, represented by the below formula, RₐM(O₂CR′)₂, wherein in M is tellurium (Te), antimony (Sb), tin (Sn) or bismuth (Bi), wherein a is 2-3, and wherein at least one of R and R′, when present, include an alkyne functional group moiety.

In embodiments, the present disclosure includes a multinuclear lithographic composition, represented by the below formula, [MₐN_{b}O_{c}H_{d}BₑR_{f}X_{g}(WCOR′)ₕ(OR″)ᵢ(WCOCOY)ⱼ(COY)ₖ]l wherein: the composition contains at least one of R, R′ and R″, and at least one of R, R′ and R″ includes an alkyne functional group moiety: R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —C≡CH, —(CO)C₆H₄C≡CH, —C≡CR′″, —CH₂C₆H₄C≡CR′″, —(CO)C₆H₄C≡CR′″, or —C₆H₄C≡CR′″-o-C₆H₄OCH₃, -m-C₆H₄OCH₃, -o-CH₂C₆H₄OCH₃, -m-CH₂C₆H₄OCH₃, -p-CH₂C₆H₄OCH₃, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH; R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, —C≡CR′″, —C₆H₄C≡CR′″, or —CH₂C₆H₄C≡CR′″-o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH; R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, or —C₆H₄C(CH₃)═CH₂, —C₆H₄C≡CH, —CH₂C₆H₄C≡CH, —C₆H₄C≡CR′″, or —CH₂C₆H₄C≡CR′″; R′″, when present, is independently an aromatic or aliphatic hydrocarbon selected from: —CH₃, —CH₂CH₃, or —C₆H₅; M is each independently an element selected from the group of tellurium (Te), antimony (Sb), tin (Sn), iodine (I), and bismuth (Bi); N, when present, is each independently an element selected from the group consisting of indium (In), tin (Sn), antimony (Sb), tellurium (Te), bismuth (Bi) and iodine (I); H, when present, is each independently hydrogen; O is each independently oxygen; B, when present, is each independently selected from W, C₂O₄, SO₄, PO₄, (CH₂)₂C₆H₂(CH₂)₂, —C≡C—, —CH₂C≡C—, —CH₂C≡CCH₂—, or —CH₂C₆H₄CH₂— —O—O—, —O₂CCH₂CH₂CO₂—, —O₂CCH═CHCO₂—, —O₂CC≡CCO₂—, —(OCO)₂NCH₂CH₂N(COO)₂—; X, when present, is each independently selected from F, Cl, Br, CN, I, or C₂O₄, an alkyne functional group moiety, —O₂CC≡CH, —O₂CC₆H₄C≡CH, or —O₂CCH₂C₆H₄C≡CH; W, when present, is each independently an element or a compound selected from the group consisting of —CH₂, NR′, S, and O; Y when present, is each independently an element or compound selected from the group consisting of —WR′, and —R′, wherein a=1-8; b=0-5; c=0-20; d=0-20; e=0-10; f=0-20; g=0-20; h=0-5; i=0-5; j=0-5; k=0-6; and I=charge on an ion or a complex selected from: −4, −3, −2, −1, 0, +1, +2, +3, +4.

In embodiments, the lithography compositions are suitable for forming radiation sensitive films such as photosensitive resists which, when exposed to light such as EUV, loses its resistance or its susceptibility to attack by an etchant or solvent. Such materials are used in making semiconductor devices such as microcircuits.

Advantages of the compositions of the present disclosure include, inter alia, improved contrast curves, improved resolution, and improved LER.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers to C₁₋₈ straight-chain alkyls. In other embodiments, “alkyl” refers to C₁₋₈ branched-chain alkyls. In embodiments, alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl. Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

As used herein, the term “heteroalkyl” by itself or in combination with another term means, unless otherwise stated, a stable straight or branched chain alkyl group consisting of the stated number of carbon atoms and one or two heteroatoms of O, N, and S, and wherein the nitrogen and sulfur atoms may be optionally oxidized and the nitrogen heteroatom may be optionally quaternized. In embodiments, the heteroatom(s) may be placed at any position of the heteroalkyl group, including between the rest of the heteroalkyl group and the fragment to which it is attached, as well as attached to the most distal carbon atom in the heteroalkyl group. Non-limiting examples include: —O—CH₂—CH₂—CH₃, —CH₂—CH₂—CH₂—OH, —CH₂—CH₂CH₂—NH—CH₃, and —CH₂—S—CH₂—CH₃. In embodiments, up to two heteroatoms may be consecutive, such as, for example, —CH₂—NH—OCH₃, or —CH₂—CH₂—S—S—CH₃. In embodiments, heteroalkyl groups have 1-12 carbons.

As used herein, the term “alkenyl,” denotes a monovalent group derived from a hydrocarbon moiety containing at least two carbon atoms and at least one carbon-carbon double bond. In embodiments, the double bond may or may not be the point of attachment to another group. Alkenyl groups (e.g., C₂-C₈-alkenyl) include, but are not limited to, for example, ethenyl, propenyl, prop-1-en-2-yl, butenyl, 1-methyl-2-buten-1-yl, heptenyl, octenyl and the like.

As used herein, the term “alkynyl,” denotes a monovalent group derived from a hydrocarbon moiety containing at least two carbon atoms and at least one carbon-carbon triple bond. In certain embodiments, the alkynyl group employed in the disclosure contains 2-20 carbon atoms. In some embodiments, the alkynyl group employed in the disclosure contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms. Representative alkynyl groups include, but are not limited to, ethynyl, 1-propynyl, 2-propynyl, 1-butynyl, 2-butynyl, 1-pentynyl, 2-pentynyl, 1-hexynyl, 2-hexynyl and the like, which may bear one or more substituents. Alkynyl group substituents include, but are not limited to, any of the substituents described herein, that result in the formation of a stable moiety. Non-limiting examples of alkynyl as used herein includes alkynyl carboxylate.

As used herein, the term “halo” or “halogen” alone or as part of another substituent means, unless otherwise stated, a fluorine, chlorine, bromine, or iodine atom.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. In embodiments, a cycloalkyl group can be optionally partially unsaturated. In embodiments, the cycloalkyl group also can be optionally substituted with an alkyl group substituent as defined herein, oxo, and/or alkylene. In embodiments, there can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Non-limiting examples of monocyclic cycloalkyl rings include cyclopentyl, cyclohexyl, and cycloheptyl. Non-limiting examples of multicyclic cycloalkyl rings include adamantyl, octahydronaphthyl, decalin, camphor, camphane, and noradamantyl.

As used herein, the term “heterocycloalkyl” or “heterocyclyl” refers to a heteroalicyclic group including one to four ring heteroatoms each selected from O, S, and N. In embodiments, each heterocyclyl group has from 3 to 10 atoms in its ring system, with the proviso that the ring of said group does not contain two adjacent O or S atoms. In embodiments, heterocyclyl substituents may be alternatively defined by the number of carbon atoms, e.g., C₂-C₈-heterocyclyl indicates the number of carbon atoms contained in the heterocyclic group without including the number of heteroatoms. For example, a C₂-C₈-heterocyclyl will include an additional one to four heteroatoms. In embodiments, the heterocyclyl group has less than three heteroatoms. In embodiments, the heterocyclyl group has one to two heteroatoms. In embodiments, the heterocycloalkyl group is fused with an aromatic ring. In embodiments, nitrogen and sulfur heteroatoms may be optionally oxidized, and the nitrogen atom may be optionally quaternized. The heterocyclic system may be attached, unless otherwise stated, at any heteroatom or carbon atom that affords a stable structure.

As used herein, the term “aromatic” refers to a carbocycle or heterocycle with one or more polyunsaturated rings and having aromatic character, i.e., having (4n+2) delocalized π (pi) electrons, where n is an integer.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. In embodiments, the term “aryl” specifically encompasses heterocyclic aromatic compounds. The aromatic ring(s) can include phenyl, naphthyl, biphenyl, diphenylether, diphenylamine and benzophenone, among others. In particular embodiments, the term “aryl” means a cyclic aromatic including about 5 to about 10 carbon atoms, e.g., 5, 6, 7, 8, 9, or 10 carbon atoms, and including 5- and 6-membered hydrocarbon and heterocyclic aromatic rings. In embodiments, an aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R and R″ can each be independently hydrogen, alkyl, substituted alkyl, aryl, substituted aryl, and aralkyl. Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto. Non-limiting examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

As generally discussed herein, a structure represented generally by the formula:

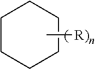

as used herein refers to a ring structure, for example, but not limited to a 3-carbon, a 4-carbon, a 5-carbon, a 6-carbon, and the like, aliphatic and/or aromatic cyclic compound, including a saturated ring structure, a partially saturated ring structure, and an unsaturated ring structure as defined herein, including a substituent R group. In embodiments, the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure above where n is 0 to 2 would comprise compound groups including, but not limited to:

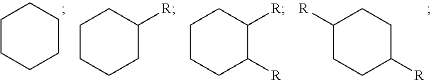

and the like.

In embodiments, a dashed line representing a bond in a cyclic ring structure indicates that the bond can be either present or absent in the ring. That is a dashed line representing a bond in a cyclic ring structure indicates that the ring structure is one of a saturated ring structure, a partially saturated ring structure, and an unsaturated ring structure.

These definitions are intended to supplement and illustrate, not preclude, the definitions that would be apparent to one of ordinary skill in the art upon review of the present disclosure. Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DESCRIPTION OF CERTAIN EMBODIMENTS

In embodiments, the present disclosure includes a mononuclear lithographic composition, represented by the formula, RₐM(O₂CR′)₂, wherein in M is tellurium (Te), antimony (Sb), tin (Sn), or bismuth (Bi), wherein a is 2-3, and wherein at least one of R and R′, include an alkyne functional group moiety. In some embodiments, M is Te, and a=2. In some embodiments, M is Sn, and a=2. In some embodiments M is Sb, and a=3. In some embodiments, M is Bi, and a=3.

In some embodiments, the present disclosure includes a mononuclear lithographic composition, represented by the formula, RₐM(O₂CR′)₂, wherein in M is tellurium (Te), antimony (Sb), tin (Sn) or bismuth (Bi), wherein a is 2-3, and wherein at least one of R and R′, include an alkyne functional group moiety. In some embodiments, R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —C≡CH, —(CO)C₆H₄C≡CH, —C≡CR′″, —CH₂C₆H₄C≡CR′″, —(CO)C₆H₄C≡CR′″, or —C₆H₄C≡CR′″-o-C₆H₄OCH₃, -m-C₆H₄OCH₃, -o-CH₂C₆H₄OCH₃, -m-CH₂C₆H₄OCH₃, -p-CH₂C₆H₄OCH₃, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH; R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, —C₆H₄C≡CH, —CH₂C₆H₄C≡CH, —C≡CR′″, —C₆H₄C≡CR′″, or —CH₂C₆H₄C≡CR′″-o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH; O is each independently oxygen; and C is each independently carbon. In embodiments, R′ is present. In embodiments, R″ is present. In embodiments, R′ and R″ are present. In some embodiments, the present disclosure includes a multinuclear lithographic composition, represented by the below formula, [MₐN_{b}O_{c}H_{d}BₑR_{f}X_{g}(WCOR′)ₕ(OR″)ᵢ(WCOCOY)ⱼ(COY)ₖ]l wherein the composition contains at least one of R, R′ and R″, and at least one of R, R′ and R″ includes an alkyne functional group moiety: R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —C≡CH, —(CO)C₆H₄C≡CH, —C≡CR′″, —CH₂C₆H₄C≡CR′″, —(CO)C₆H₄C≡CR′″, or —C₆H₄C≡CR′″-o-C₆H₄OCH₃, -m-C₆H₄OCH₃, -o-CH₂C₆H₄OCH₃, -m-CH₂C₆H₄OCH₃, -p-CH₂C₆H₄OCH₃, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH; R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, —C≡CR′″, —C₆H₄C≡CR′″, or —CH₂C₆H₄C≡CR′″-o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH; R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, or —C₆H₄C(CH₃)═CH₂, —C₆H₄C≡CH, —CH₂C₆H₄C≡CH, —C₆H₄C≡CR′″, or —CH₂C₆H₄C≡CR′″; R′″, when present, is independently an aromatic or aliphatic hydrocarbon selected from: —CH₃, —CH₂CH₃, or —C₆H₅; however, at least one of R and R′, include an alkyne functional group moiety; M is each independently an element selected from the group of tellurium (Te), antimony (Sb), tin (Sn), iodine (I), bismuth (Bi) and indium (In); N, when present, is each independently an element selected from the group consisting of indium (In), tin (Sn), antimony (Sb), tellurium (Te), bismuth (Bi) and iodine (I); H, when present, is each independently hydrogen; O is each independently oxygen; B, when present, is each independently selected from W, C₂O₄, SO₄, PO₄, (CH₂)₂C₆H₂(CH₂)₂, —C≡C—, —CH₂C≡C—, —CH₂C≡CCH₂—, or —CH₂C₆H₄CH₂— —O—O—, —O₂CCH₂CH₂CO₂—, —O₂CCH═CHCO₂—, —O₂CC≡CCO₂—, —(OCO)₂NCH₂CH₂N(COO)₂—; X, when present, is each independently selected from F, Cl, Br, CN, I, or C₂O₄, an alkyne functional group moiety, —O₂CC≡CH, —O₂CC₆H₄C≡CH, or —O₂CCH₂C₆H₄C≡CH; W, when present, is each independently an element or a compound selected from the group consisting of —CH₂, NR′, S, and O; Y when present, is each independently an element or compound selected from the group consisting of —WR′, and —R′, wherein a=1-8; b=0-5; c=0-20; d=0-20; e=0-10; f=0-20; g=0-20; h=0-5; i=0-5; j=0-5; k=0-6; and I=charge on an ion or a complex selected from: −4, −3, −2, −1, 0, +1, +2, +3, +4.

In some embodiments, X is present and

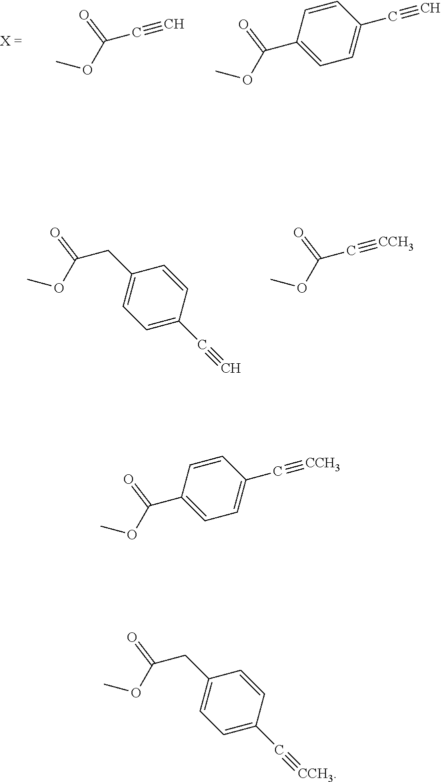

In some embodiments, R′ is present and

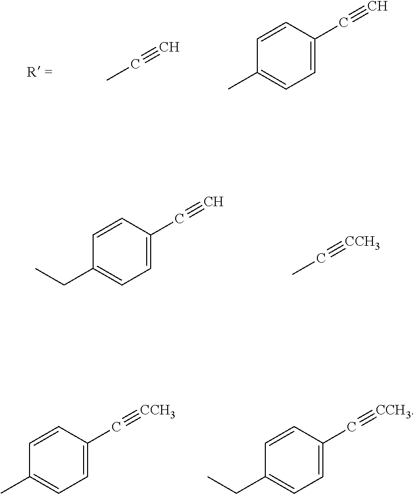

In some embodiments, R is present, and

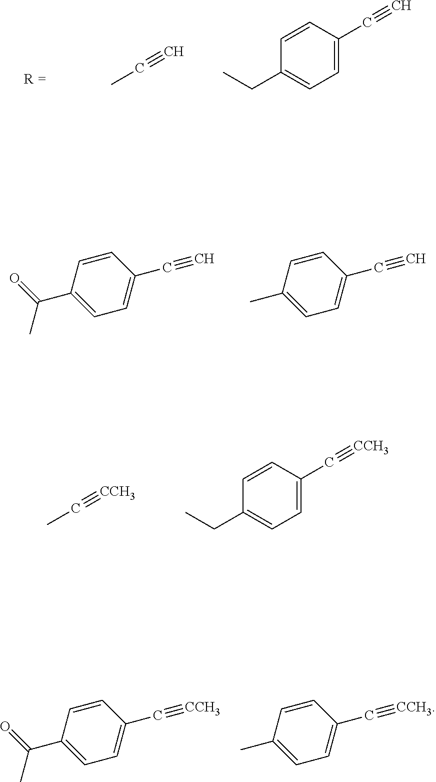

In embodiments, the compositions are suitable for use as EUV photoresist films, deposited atop a substrate in an electronic device manufacturing process, or semiconductor manufacturing process flow. In embodiments, it is understood that if a value in the formula is zero (0), then the constituent is not present in the composition.

It is also to be understood that the compositions and/or lithographic compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

In embodiments, the present disclosure includes a lithographic composition represented by the below formula, RₐM(O₂CR′)₂, wherein in M is tellurium (Te), antimony (Sb), tin (Sn), or bismuth (Bi), wherein a is 2-3, and wherein at least one of R and R′, when present, include an alkyne functional group moiety. In embodiments, the present disclosure includes a lithographic composition, represented by the below formula, [MₐN_{b}O_{c}H_{d}BₑR_{f}X_{g}(WCOR′)ₕ(OR″)ᵢ(WCOCOY)ⱼ(COY)ₖ]l wherein the composition contains at least one of R, R′ and R″, and at least one of R, R′ and R″ includes an alkyne functional group moiety: R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —C≡CH, —(CO)C₆H₄C≡CH, —C≡CR′″, —CH₂C₆H₄C≡CR′″, —(CO)C₆H₄C≡CR′″, or —C₆H₄C≡CR′″: -o-C₆H₄OCH₃, -m-C₆H₄OCH₃, -o-CH₂C₆H₄OCH₃, -m-CH₂C₆H₄OCH₃, -p-CH₂C₆H₄OCH₃, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH; R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, —C≡CR′″, —C₆H₄C≡CR′″, or —CH₂C₆H₄C≡CR′″-o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH; R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, or —C₆H₄C(CH₃)═CH₂, —C₆H₄C≡CH, —CH₂C₆H₄C≡CH, —C₆H₄C≡CR′″, or —CH₂C₆H₄C≡CR′″; R′″, when present, is independently an aromatic or aliphatic hydrocarbon selected from: —CH₃, —CH₂CH₃, or —C₆H₅; M is each independently an element selected from the group of tellurium (Te), antimony (Sb), tin (Sn), iodine (I), and bismuth (Bi); N, when present, is each independently an element selected from the group consisting of indium (In), tin (Sn), antimony (Sb), tellurium (Te), bismuth (Bi) and iodine (I); H, when present, is each independently hydrogen; O is each independently oxygen; B, when present, is each independently selected from W, C₂O₄, SO₄, PO₄, (CH₂)₂C₆H₂(CH₂)₂, —C≡C—, —CH₂C≡C—, —CH₂C≡CCH₂—, or —CH₂C₆H₄CH₂—, —O—O—, —O₂CCH₂CH₂CO₂—, —O₂CCH═CHCO₂—, —O₂CC≡CCO₂—, —(OCO)₂NCH₂CH₂N(COO)₂—; X, when present, is each independently selected from F, Cl, Br, CN, I, or C₂O₄, an alkyne functional group moiety, —O₂CC≡CH, —O₂CC₆H₄C≡CH, or —O₂CCH₂C₆H₄C≡CH; W, when present, is each independently an element or a compound selected from the group consisting of —CH₂, NR′, S, and O; Y when present, is each independently an element or compound selected from the group consisting of —WR′, and —R′, wherein a=1-8; b=0-5; c=0-20; d=0-20; e=0-10; f=0-20; g=0-20; h=0-5; i=0-5; j=0-5; k=0-6; and I=charge on an ion or a complex selected from: −4, −3, −2, −1, 0, +1, +2, +3, +4. In certain embodiments, the multinuclear compositions are devoid of tin (Sn).

In embodiments, the present disclosure includes a film composition represented by the below formula, RₐM(O₂CR′)₂, wherein in M is tellurium (Te), antimony (Sb), tin (Sn), or bismuth (Bi), wherein a is 2-3, and wherein at least one of R and R′, when present, include an alkyne functional group moiety.

In embodiments, the present disclosure includes a film composition, represented by the below formula, [MₐN_{b}O_{c}H_{d}BₑR_{f}X_{g}(WCOR′)ₕ(OR″)ᵢ(WCOCOY)ⱼ(COY)ₖ]l wherein: the composition contains at least one of R, R′ and R″, and at least one of R, R′ and R″ includes an alkyne functional group moiety: R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —C≡CH, —(CO)C₆H₄C≡CH, —C≡CR′″, —CH₂C₆H₄C≡CR′″, —(CO)C₆H₄C≡CR′″, or —C₆H₄C≡CR′″-o-C₆H₄OCH₃, -m-C₆H₄OCH₃, -o-CH₂C₆H₄OCH₃, -m-CH₂C₆H₄OCH₃, -p-CH₂C₆H₄OCH₃, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH; R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, —C≡CR′″, —C₆H₄C≡CR′″, or —CH₂C₆H₄C≡CR′″-o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH; R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, or —C₆H₄C(CH₃)═CH₂, —C₆H₄C≡CH, —CH₂C₆H₄C≡CH, —C₆H₄C≡CR′″, or —CH₂C₆H₄C≡CR′″; R′″, when present, is independently an aromatic or aliphatic hydrocarbon selected from: —CH₃, —CH₂CH₃, or —C₆H₅; M is each independently an element selected from the group of tellurium (Te), antimony (Sb), tin (Sn), or bismuth (Bi); N, when present, is each independently an element selected from the group consisting of indium (In), antimony (Sb), tellurium (Te), bismuth (Bi) and iodine (I); H, when present, is each independently hydrogen; O is each independently oxygen; B, when present, is each independently selected from W, C₂O₄, SO₄, PO₄, (CH₂)₂C₆H₂(CH₂)₂, —C≡C—, —CH₂C≡C—, —CH₂C≡CCH₂—, or —CH₂C₆H₄CH₂—, —O—O—, —O₂CCH₂CH₂CO₂—, —O₂CCH═CHCO₂—, —O₂CC≡CCO₂—, —(OCO)₂NCH₂CH₂N(COO)₂—;
X, when present, is each independently selected from F, Cl, Br, CN, I, or C₂O₄, an alkyne functional group moiety, —O₂CC≡CH, —O₂CC₆H₄C≡CH, or —O₂CCH₂C₆H₄C≡CH; W, when present, is each independently an element or a compound selected from the group consisting of —CH₂, NR′, S, and O; Y when present, is each independently an element or compound selected from the group consisting of —WR′, and —R′, wherein a=1-8; b=0-5; c=0-20; d=0-20; e=0-10; f=0-20; g=0-20; h=0-5; i=0-5; j=0-5; k=0-6; and I=charge on an ion or a complex selected from: −4, −3, −2, −1, 0, +1, +2, +3, +4.

In embodiments, the present disclosure includes a method for forming a radiation patternable coating, the method including: contacting a coating solution of the present disclosure with a substrate under conditions suitable for forming a film atop the substrate. In embodiments, the substrate is heated to a temperature from about 30 degrees Celsius to about 250 degrees Celsius for about 0.5 minutes to about 30 minutes. In embodiments, the coating solution is spin coated to form a film atop the substrate.

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where a chiral center exists in a structure, if any, but no specific stereochemistry is shown for that center, both enantiomers, separately or as a mixture, are encompassed by that structure. The methods for the determination of stereochemistry and the separation of stereoisomers are well-known in the art.

In embodiments, any polycyclic compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Non-limiting examples of isotopes suitable for inclusion in the compounds described herein include and are not limited to ²H or deuterium. In one embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In embodiments, the compounds described herein are labeled by other means, including, but not limited to, the use of chromophores or fluorescent moieties, bioluminescent labels, or chemiluminescent labels.

In embodiments, the compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein. Non-limiting examples of forming one or more compounds of the present disclosure are described in the Example section below; however, in embodiments, compounds described herein are synthesized using any suitable procedures starting from compounds that are available from commercial sources or are prepared using procedures described herein.

In embodiments, the present disclosure relates to a method for forming a resist pattern using a lithography composition, including contacting a substrate with a lithography composition of the present disclosure to form a thin radiation sensitive film of the lithography composition atop the substrate.

In embodiments, the present disclosure relates to a method for forming a semiconductor device using one or more lithography compositions of the present disclosure in a photolithography method of the present disclosure. In embodiments, a method for forming a semiconductor device includes contacting a substrate with a lithography composition of the present disclosure to form a thin radiation sensitive film of the lithography composition atop the substrate.

In embodiments, the present disclosure relates to a method for forming an electronic device using the lithography compositions in a photolithography method of the present disclosure. In embodiments, a method for forming an electronic device includes contacting a substrate with a lithography composition of the present disclosure to form a thin radiation sensitive film of the lithography composition atop the substrate.

In embodiments, the present disclosure relates to a substrate and a radiation sensitive coating including a composition of the present disclosure.

In embodiments, the present disclosure related to a substrate including an inorganic semiconductor layer and a radiation sensitive coating material atop a surface, wherein the radiation sensitive coating includes a lithographic composition of the present disclosure. In some embodiments, the radiation coating material or film of the present disclosure can be patterned with EUV light at a wavelength of 13.5 nm in a pattern of 16-nm lines, with a line width roughness of no more than about 4 nm. In embodiments, the radiation sensitive coating material can include metal, such as Te, and can include at least 5 weight percent metal and in other embodiments at least about 20 weight percent metal.

In embodiments, a method for forming an electronic device such as a semiconductor device using the lithography compositions in a photolithography method of the present disclosure is provided. In embodiments, a method for forming an electronic device such as a semiconductor device includes contacting a substrate with a lithography composition of the present disclosure to form a thin radiation sensitive film of the lithography composition atop the substrate. In embodiments, the radiation sensitive film is characterized as amorphous. In embodiments, the radiation sensitive film has a thickness of 10-50 nanometers, such as 20-40 nanometers, such as about 20 nanometers, or 20 nanometers.

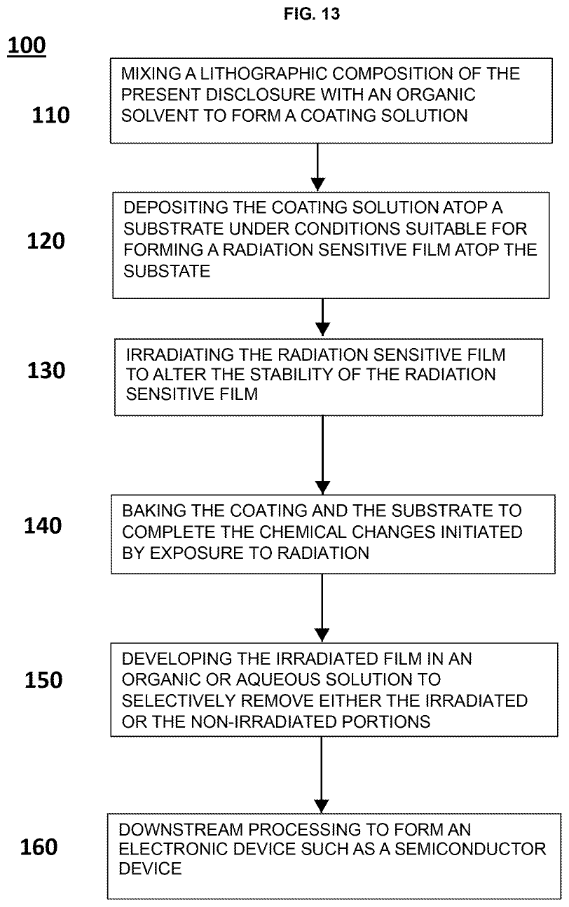

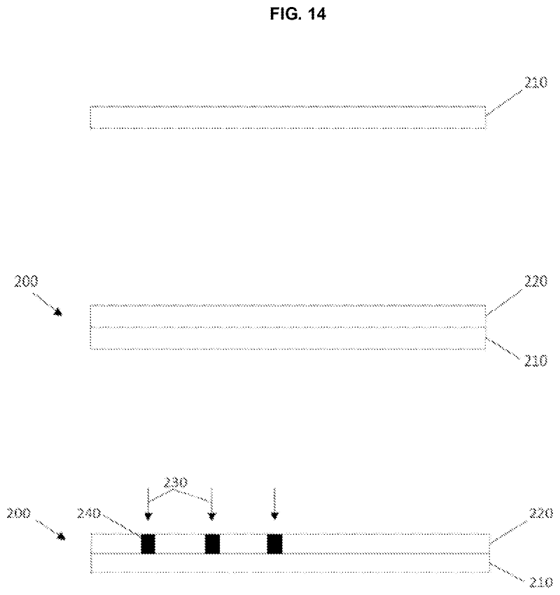

FIG. 13 depicts a flow diagram illustrating a process 100 for forming an electronic device such as a semiconductor device using the lithography compositions in a photolithography method of the present disclosure, which corresponds to FIGS. 14A-14C illustrating schematic cross-sectional views of electronic device 200 such as a semiconductor device at different stages of fabrication. In some embodiments, process 100 is a process flow, and operations 110, 120, 130, 150 and optionally 140 and 160 are individual processes. The process 100 is configured to be performed in an electronic device or semiconductor device manufacturing facility using equipment suitable for depositing layers or coatings atop a substrate.

In embodiments, the process 100 may begin at operation 110 mixing a lithographic composition of the present disclosure with an organic or aqueous solvent to form a coating solution. For example, a lithographic composition of the present disclosure is formed and provided, and subsequently formed into a solution including a lithographic composition of the present disclosure, including one or more lithographic compounds described above having a general formula RₐM(O₂CR′)₂, or a multinuclear lithographic composition, represented by the below formula, [MₐN_{b}O_{c}H_{d}BₑR_{f}X_{g}(WCOR′)ₕ(OR″)ᵢ(WCOCOY)ⱼ(COY)ₖ]l (as described hereinabove), and an organic or aqueous solvent. In embodiments, the lithographic composition of the present disclosure is provided in an amount sufficient to form a coating solution suitable for forming a film having a predetermined thickness over a substrate.

In embodiments, and still referring to FIG. 13, the process 100 may begin at operation 110 by mixing a lithographic composition of the present disclosure with an organic or aqueous solvent to form a coating solution. In embodiments, the solvent is an alcohol, an ester, or a mixture thereof. In embodiments, a predetermined lithographic composition can be dissolved in an organic solvent, e.g., alcohols, aromatic and aliphatic hydrocarbons, esters or combinations thereof. Non-limiting examples of suitable solvents include, for example, aromatic compounds (e.g., xylenes, toluene), ethers (anisole, tetrahydrofuran), esters (propylene glycol monomethyl ether acetate, ethyl acetate, ethyl lactate), ethers (propylene glycol monomethyl ether acetate), alcohols (e.g., 4-methyl-2-propanol, 1-butanol, methanol, isopropyl alcohol, 1-propanol), ketones (e.g., methyl ethyl ketone), halogenated hydrocarbons (e.g., 1,2-dichloroethane, chlorobenzene), mixtures thereof, and the like. In embodiments, organic solvent selection can be influenced by solubility parameters, volatility, flammability, toxicity, viscosity and potential chemical interactions with other processing materials. After the components of the solution are dissolved and combined, the character of the species may change as a result of partial in-situ hydrolysis, hydration, and/or condensation. When the composition of the solution is referenced herein, the reference is to the components as added to the solution, since complex formulations may undergo solvolysis and ligand metathesis, or produce metal polynuclear species in solution that may not be well characterized.

In embodiments, a substrate 210 is provided in the form of silicon, glass, metal, or any suitable material for use as a substrate in electronic device or semiconductor device manufacturing.

Referring to FIG. 13, process 100 includes at process sequence 120 depositing the coating solution atop a substrate under conditions suitable for forming a radiation sensitive film atop the substrate. In embodiments, conditions suitable for forming a radiation sensitive film atop the substrate include conditions suitable for removing the solvent to form a film of lithographic composition in accordance with the present disclosure. In embodiments, removing the solvent may be performed by any method known in the art including heating the mixture under conditions sufficient to evaporate the solvent. For example, the mixture can be spin-coated by depositing 0.5-5 mL of the mixture on a silicon wafer, then spinning the wafer from 500-10,000 rpm for 30-60 seconds. In embodiments, after removing the solvent, a coating layer 220 or film including lithographic composition of the present disclosure is formed and disposed atop substrate 210, as shown in FIG. 14B such as wherein the substrate is formed of a preselected materials such as silicon, glass, metal, or the like as described above. In some embodiments, and depending upon needs, the solvent is removed under conditions which permit the formation of an amorphous film of lithographic composition of the present disclosure, wherein the coating layer 220 is characterized as a lithographic film which is photosensitive or reactive to EUV.

Referring back to FIG. 13, process 100 at process sequence 130 optionally includes irradiating the radiation sensitive film such as coating layer 220 to alter the stability of the radiation sensitive film. In embodiments, the lithographic composition in the form of a film is subject to radiation such as EUV to promote or initiate a chemical change in the irradiated portions. In embodiments, a radiation process is applied under conditions suitable to penetrate radiation such as EUV into the coating layer 220 such as a film layer. For example, referring to FIG. 14C, radiation (shown as arrows 230) is applied in an amount and under conditions to alter one or more exposed regions of coating layer 220 atop the substrate 210 to form unstable regions 240. In embodiments, the radiation process is applied for a duration sufficient to penetrate the entire height of the coating layer or film. Regions next to or immediately adjacent the one or more exposed regions may be characterized as unexposed regions. In embodiments, the radiation alters a predetermined portion of the coating layer or film of the present disclosure.

Referring back to FIG. 13, in embodiments, process 140 optionally includes a bake step from 40-220° C. for 30-120 s to complete the chemical transformation initiated by the irradiation process 130.

Continuing with FIG. 13, in process 150, the irradiated film is immersed in an organic or aqueous solution or pure solvent. The altered portion may be removed or left behind depending upon process needs.

In embodiments, the coating compositions for forming the resist coatings generally include organometallic compositions of the present disclosure with appropriate radiation sensitive characteristics. For processing into a patternable coating, the lithographic compositions and ligands described herein, are generally formed into a solution with a solvent, generally an organic or aqueous solvent that can be formed into a coating through solution coating or a vapor-based deposition process. The ultimate resist coatings are based on organometallic chemistry, and the lithographic compositions of the present disclosure provide stable solutions with good resist properties. In embodiments, one or more ligands are generally selected to facilitate solution formation and related processing functions. In embodiments, lithographic compositions of the present disclosure with a ligand of the present disclosure can be introduced as a solution to improve the range of compositions that can be formed into stable solutions with the expectation that the coating can provide for patternable coatings with organometallic materials. Compositions of the present disclosure provide desirable patterning properties.

In embodiments, the concentrations of the organometallic materials in the solutions can be selected to achieve desired physical properties of the solution. In particular, lower concentrations overall can result in desirable properties of the solution for certain coating approaches, such as spin coating, that can achieve thinner coatings using reasonable coating parameters. It can be desirable to use thinner coatings to achieve ultrafine patterning. In general, the concentration can be selected to be appropriate for the selected coating approach.

In embodiments, coating layer 220 may be formed through deposition and subsequent processing onto a selected substrate. Using the lithographic compositions and coating compositions described herein, some hydrolysis and condensation generally is performed during coating, and may be completed or furthered post coating via subsequent processing steps such as heating in air. In embodiments, a substrate such as substrate 210 generally presents a surface onto which the coating material can be deposited, and the substrate 210 may include a plurality of layers in which the surface relates to an upper most layer. In some embodiments, the substrate surface can be treated to prepare the surface for adhesion of the coating material. Also, the surface can be cleaned and/or smoothed as appropriate. Suitable substrate surfaces may include any reasonable material. Some substrates of particular interest include, for example, silicon wafers, silica substrates, other inorganic materials such as ceramic materials, polymer substrates, such as organic polymers, composites thereof and combinations thereof across a surface and/or in layers of the substrate. Wafers, such as relatively thin cylindrical structures, can be convenient, although any reasonable shaped structure can be used. Polymer substrates or substrates with polymer layers on non-polymer structures can be desirable for certain applications based on their low cost and flexibility, and suitable polymers can be selected based on the relatively low processing temperatures that can be used for the processing of the patternable materials described herein.

In some embodiments, where patterning is performed using radiation, spin coating is a suitable approach to cover the substrate relatively uniformly, although there can be edge effects. In some embodiments, a wafer can be spun at rates from about 400 rpm to about 10,000 rpm. The spinning speed can be adjusted to obtain a desired coating thickness. The spin coating can be performed for times from about 5 seconds to about 5 minutes and in further embodiments from about 15 seconds to about 2 minutes. An initial low speed spin, e.g., at 50 rpm to 250 rpm, can be used to perform an initial bulk spreading of the composition across the substrate. A back side rinse, edge bead removal step or the like can be performed with water or other suitable solvent to remove any edge bead. A person or ordinary skill in the art will recognize that additional ranges of spin coating parameters within the explicit ranges above are contemplated and are within the present disclosure.

The thickness of the coating generally can be a function of the coating solution concentration, viscosity and the spin speed for spin coating. For other coating processes, the thickness can generally also be adjusted through the selection of the coating parameters. In some embodiments, it can be desirable to use a thin coating to facilitate formation of small and highly resolved features in the subsequent patterning process. For example, the coating materials after drying can have an average thickness of no more than about 1 micron, in further embodiments no more than about 250 nanometers (nm), in additional embodiments from about 1 nanometers (nm) to about 50 nm, in other embodiments from about 2 nm to about 40 nm and in some embodiments from about 3 nm to about 25 nm.

In embodiments, the coating process itself can result in the evaporation of a portion of the solvent since many coating processes form droplets or other forms of the coating material with larger surface areas and/or movement of the solution that stimulates evaporation. The loss of solvent tends to increase the viscosity of the coating material as the concentration of the species in the material increases. An objective during the coating process can be to remove sufficient solvent to stabilize the coating material for further processing. Reactive species may condense during coating or subsequent heating to forming a coating material.

Following drying, the coating material can be finely patterned using radiation. In embodiments, the absorption of the radiation results in energy that can break the bonds between the metal and alkyl or carboxylate ligands so that at least some of the alkyl ligands are no longer available to stabilize the material. Radiolysis products, including CO₂, alkyl ligands or fragments may diffuse out of the film, or not, depending on process variables and the identity of such products. In embodiments, with the absorption of a sufficient amount of radiation, the exposed coating material condenses. The radiation generally can be delivered according to a selected pattern. In embodiments, the radiation pattern is transferred to a corresponding pattern or latent image in the coating material with irradiated areas and un-irradiated areas. The irradiated areas include chemically altered coating material, and the un-irradiated areas include generally the as-formed coated material. As noted below, very smooth edges can be formed upon development of the coating material with the removal of the un-irradiated coating material or alternatively with selective removal of the irradiated coating material.

Radiation generally can be directed to the coated substrate through a mask or a radiation beam and controllably scanned across the substrate. In embodiments, the radiation can include electromagnetic radiation, an electron-beam (beta radiation), or other suitable radiation. In embodiments, electromagnetic radiation can have a desired wavelength or range of wavelengths, such as visible radiation, ultraviolet radiation or x-ray radiation. The resolution achievable for the radiation pattern is generally dependent on the radiation wavelength, and a higher resolution pattern generally can be achieved with shorter wavelength radiation. Thus, it can be desirable to use ultraviolet light, x-ray radiation or an electron-beam to achieve particularly high-resolution patterns.

In embodiments, ultraviolet light is provided which extends between wavelengths of greater than or equal 100 nm and less than 400 nm. In embodiments, a krypton fluoride laser can be used as a source for 248 nm ultraviolet light. The ultraviolet range can be subdivided in several ways, such as extreme ultraviolet (EUV) from greater than or equal 10 nm to less than 121 nm and far ultraviolet (FUV) from greater than or equal to 122 nm to less than 200 nm. In embodiments, EUV light has been used for lithography at 13.5 nm, and this light is generated from a Xe or Sn plasma source excited using high energy lasers or discharge pulses.

The amount of electromagnetic radiation can be characterized by a fluence or dose which is defined by the integrated radiative flux over the exposure time. A person of ordinary skill in the art will recognize the ranges of radiation fluences within the explicit ranges above are contemplated and are within the present disclosure.

Based on the design of the coating material, there can be a large contrast of material properties between the irradiated regions that have condensed coating material and the un-irradiated, coating material with substantially intact organic ligands.

In embodiments, following exposure with radiation, the coating material is patterned with irradiated regions and un-irradiated regions. Referring to FIG. 14C, a patterned structure is shown including a substrate 210, a thin film 220 and patterned coating material 240 or regions of instability. In embodiments, patterned coating material includes region 240 of irradiated coating material and uncondensed regions un-irradiated coating material. The pattern formed by condensed regions and uncondensed regions represent a latent image into the coating material, and the development of the latent image is performed as is known in the art.

In embodiments, development of the image involves the contact of the patterned coating material including the latent image to a developer composition to remove either the un-irradiated coating material to form the negative image or the irradiated coating to form the positive image. Using the resist materials described herein, effective negative patterning or positive patterning generally can be performed with desirable resolution using appropriate developing solutions, and generally based on the same coating. In particular, the irradiated regions are at least partially condensed to increase the metal oxide character so that the irradiated material is resistant to dissolving by organic solvents while the un-irradiated compositions remain soluble in the organic solvents. Reference to a condensed coating material refers to at least partial condensation in the sense of increasing the oxide character of the material relative to an initial material. On the other hand, the un-irradiated material is less soluble in weak aqueous bases or acids due to the hydrophobic nature of the material so that aqueous bases can be used to remove the irradiated material while maintaining the non-irradiated material for positive patterning.

Based on the improved process described in the Examples below, the improved properties of the coating material can be correspondingly characterized. For example, a substrate including an inorganic semiconductor layer and a radiation sensitive coating material of the present disclosure along a surface can be subjected to patterning with EUV light at a wavelength of 13.5 nm in a pattern of 16-nm lines on a 32-nm pitch. In embodiments, a surface can be subjected to patterning with EUV light at a wavelength of 13.5 nm in a pattern of 32-nm lines on a 64-nm pitch. To evaluate the coating material the dose to achieve a critical dimension of 16 nm can be evaluated along with the achievable line width roughness (LWR). In embodiments, the coatings can achieve a critical dimension of 16 nm with a dose from about 20 mJ/cm2 to about 120 mJ/cm2 with a line width roughness of no more than about 4 nm. Resist critical dimension (CD) and line-width-roughness (LWR) were extracted from SEM images.

In further embodiments, the improved patterning capability can be expressed in terms of the dose-to-gel value. A structure comprising a substrate and a radiation sensitive coating comprising an alkyl metal oxide hydroxide can have a dose-to-gel (D_{g}) of no more than about 60 mJ/cm² and in further embodiments from about 10 mJ/cm² to about 40 mJ/cm². Evaluation of dose-to-gel is explained in the Examples below.

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present disclosure.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### Example I

Alkynyl Ligands for Mononuclear and Multinuclear Metal-Containing EUV Resists: Since 2009, the photoresist community has shown a great deal of interest in EUV photoresists containing metallic elements. Incorporation of metals may enhance performance due to high absorptivity, small molecular volume, high material homogeneity and high etch resistance. In 2011 Inpria and the Ober group at Cornell University described the use of hafnium oxide as a photoresist for 193-nm and EUV lithography. Later, the Ober group extensively explored the use of photoresists prepared from HfO₂ and ZrO₂ nanoparticles. Improvements in resolution, LER and sensitivity were possible by studying the effects of ligand structure and the overall patterning mechanism.

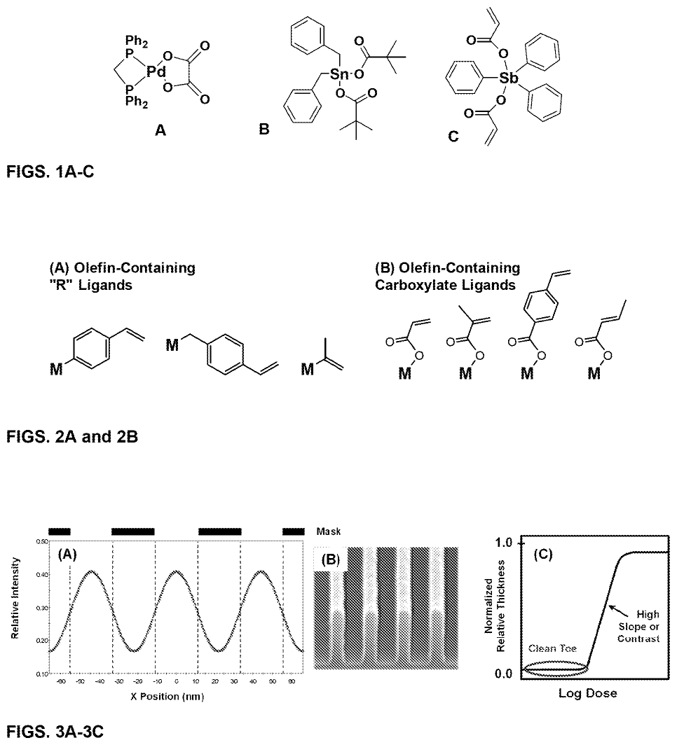

Since 2011, the Brainard Group has developed EUV photoresists composed of amorphous thin-films of compounds containing tin, cobalt, platinum, palladium, bismuth and antimony in a project called Molecular Organometallic Resists for EUV (MORE) (See e.g., FIG. 1). Referring now to FIGS. 1A-1C, three mononuclear MORE photoresists are shown. Each of these compounds exhibit unique performance characteristics such as (FIG. 1A) positive-tone imaging by palladium oxalates, (FIG. 1B) extremely low line-edge roughness by tin carboxylates and (FIG. 1C) extraordinary photospeed by antimony carboxylates that contain olefins.

Ligands Containing Polymerizable Olefins: Some of the most useful ligands used in MORE resists have contained polymerizable olefin groups. The use of olefins was explored in five-coordinate antimony and bismuth complexes of the type R₃M(O₂CR′)₂ where M=Sb or Bi, and R or R′ is an organic radical. The use of olefins was also explored in four-coordinate tellurium and tin complexes of the type R₂M(O₂CR′)₂ where M=Te or Sn, and R or R′ is an organic radical. Overall, the use of three R ligands and four carboxylate ligands (see e.g., FIGS. 2A and 2B) were explored. FIGS. 2A and 2B depict olefin-containing ligands. FIG. 2A depicts R ligands and FIG. 2B depicts carboxylate ligands.

Non-Linear Contrast Curves: High-resolution photoresists must convert aerial images (light intensity as a function of position, FIG. 3A) into high-aspect ratio three-dimensional images (FIG. 3B). The shape of a resist's contrast curve gives a great deal of information about how well a resist will convert aerial images into high resolution images. An ideal contrast curve will not respond to low doses (have a clean toe), but will show a dramatic change in solubility once a threshold dose is reached. The slope of the linear portion of the curve (FIGS. 3A-3B) provides a measure of how dramatically the solubility changes with dose, with higher slopes being better. More specifically, FIG. 3A depicts an aerial image of dense lines and spaces. FIG. 3B depicts a high-aspect ratio resist profile. FIG. 3C depicts an ideal, non-linear contrast curve for a negative-tone resist. Resists must not respond to low doses, but then exhibit a dramatic change in solubility once a threshold dose is reached.

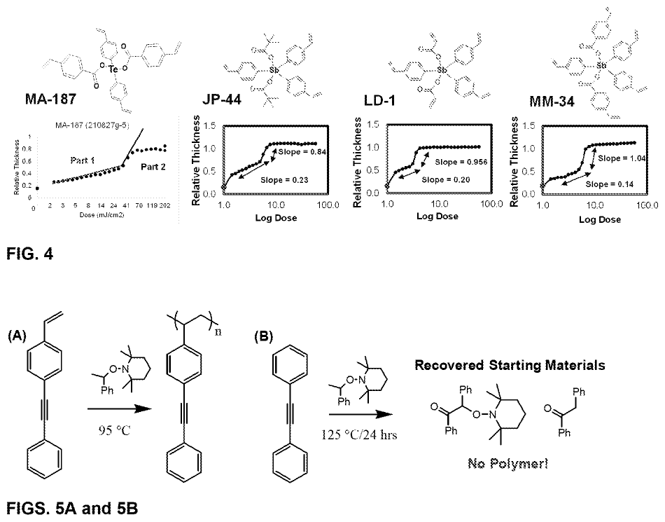

A problem encountered with many of the olefin-containing resists is that they show a two-part curve, with a high-speed response yielding a low slope, followed by a second part with higher contrast (FIG. 4). More specifically, FIG. 4 depicts negative-tone contrast curves for one tellurium and three antimony olefin-containing resists are shown.

Alkynes do not Readily Undergo Free-Radical Polymerization: Although numerous MORE compounds have been made containing olefins, specifically with the idea that the olefins would participate in free-radical polymerization, the use of alkynes (molecules containing carbon-carbon triple bonds) has not been explored as a variation to the olefins (molecules that contain carbon-carbon double bonds). This is because alkynes do not readily undergo free radical polymerization. For example, Sessions et al., demonstrated that they could polymerize an olefin in the presence of an alkyne (FIG. 5A), but under more stringent conditions they found evidence of low levels of crosslinking which the authors attributed to reactions of the alkyne side groups with radicals. Additionally, the authors set up a model study to determine how reactive alkynes are by themselves (FIG. 5B). They reacted diphenylacetylene (DPA) under free-radical conditions at 125° C. for 24 hours, and recovered 77% of the DPA, a variety of small molecules, but no polymer. More specifically, FIG. 5A depicts alkenes polymerize in the presence of alkyne. In FIG. 5B, no polymer is produced from the reaction of DPA under free-radical conditions at high temperature.

Procedures for The Synthesis: All reactions were carried out under a nitrogen environment. The intermediates dibenzylltin dibromide, Triphenyltin chloride, potassium acrylate, and potassium propynoate were made by members of our group. All other reagents were purchased from Sigma Aldrich. All reagents were used as received unless specified. Dibenzyltin dibromide was prepared through a literature procedure.

Synthetic Procedure for Dibenzyltin Diacrylate (MA-258): The general procedure was adapted from procedures found in the chemistry literature. 460 mg of R₂MX₂ (R=benzyl; M=Tin; X=Br) (1 mmol) was combined with 220 mg of the potassium acrylate (2 mmol) in an emulsion mixture of dichloromethane and water with a 2:1 ratio (20 mL:10 mL) in a 50-mL round-bottom flask, equipped with a stir bar and reflux condenser. The mixture was refluxed for 24 hours, cooled, and the phases were separated using an aqueous workup. The organic phase was evaporated to dryness at reduced pressure to a white powder and was used as is.

Synthetic Procedure for Dibenzyltin Dipropyonate (MA-257): The general procedure was adapted from procedures found in the chemistry literature. 460 mg of R₂MX₂ (R=benzyl; M=Tin; X=Br) (1 mmol) was combined with 216 mg of the potassium propyonate (2 mmol) in an emulsion mixture of dichloromethane and water with a 2:1 ratio (20 mL:10 mL) in a 50-mL round-bottom flask, equipped with a stir bar and reflux condenser. The mixture was stirred at refluxed for 24 hours, cooled, and the phases were separated using an aqueous workup. The organic phase was evaporated to dryness at reduced pressure to a white powder and was used as is.

Synthetic Procedure for Triphenyl antimony Dipropyonate (MN2-113): The general procedure was adapted from procedures found in the chemistry literature. 512 mg of R₃MX₂ (R=Ph; M=Sb; X=Br) (1 mmol) was combined with 216 mg of the potassium propyonate (2 mmol) in an emulsion mixture of dichloromethane and water with a 2:1 ratio (20 mL:10 mL) in a 50-mL round-bottom flask, equipped with a stir bar and reflux condenser. The mixture was stirred at refluxed for 24 hours, cooled, and the phases were separated using an aqueous workup. The organic phase was evaporated to dryness at reduced pressure to a white powder and was used as is.

Synthetic Procedure for Triphenyl antimony Diacrylate (JP-20/MN2-41): The general procedure was adapted from procedures found in the chemistry literature. 512 mg of R₃MX₂ (R=Ph; M=Sb; X=Br) (1 mmol) was combined with 220 mg of the potassium acrylate (2 mmol) in an emulsion mixture of dichloromethane and water with a 2:1 ratio (20 mL:10 mL) in a 50-mL round-bottom flask, equipped with a stir bar and reflux condenser. The mixture was refluxed for 24 hours, cooled, and the phases were separated using an aqueous workup. The organic phase was evaporated to dryness at reduced pressure to a white powder and was used as is.

The Synthetic Procedure for Bis-(Diphenyltin propyonate) ethyl (MA-297): Step 1: [Bis (Triphenylstin) Ethyl]. Triphenyltin Chloride (1.0 equivalent, 5 g) was added to a 2 neck round bottom flask (250 mL) followed a magnetic stir bar. The flask was then sealed with a septa and a glass stopper. The entire setup was then purged with nitrogen for 2 hours to keep the reaction conditions as dry (free of moisture) as possible. Dry tetrahydrofuran (100 mL) was then added through the septa via syringe and stirring was applied for 5 mins to dissolve the solid. High pressure nitrogen was applied through the septa and the glass stopper was removed to add pellets of sodium metal (2.5 equiv, 745 mg) to the flask. The flask was then resealed with the glass stopper and the high-pressure nitrogen was removed from the flask. The mixture was stirred for 24 hours at room temperature. While stirring, ethylene glycol (0.5 equiv, 0.73 ml) was added dropwise. The mixture stirred for an additional 15 minutes. After the reaction ended, the excess sodium was carefully removed and placed on isopropyl alcohol to quench. After the reaction was completed, the mixture was filtered, and the solvent was removed through rotary evaporation. Water (100 mL), ether (100 mL) and benzene (50 mL) were added to dissolve the residue and was placed in a separatory funnel. The combined organic layers were collected, separated, and dried with anhydrous sodium sulfate. The solvent was distilled off under reduced pressure, and white powder was obtained. This compound is stable to air in the solid state but oxidizes slowly in solution.

Step 2: [Bis (Diiphenyltin Iodide) Ethyl]. Bis (Triphenyltin) Ethyl (1.0 equiv, 0.728 mg) was added to a round bottom flask (100 mL) with a magnetic stir bar followed by benzene (70 mL). The flask was then sealed with a septum. While applying stirring at room temperature, a solution of iodine (2.0 equiv, 1.345 g) in methanol (20 mL) was added via syringe, the mixture stirred for 48 hours. After the reaction was completed, the mixture was filtered, and the solvent was removed through rotary evaporation which made a slightly pink powder.

Step 3: [Bis (Diiphenyltin Iodide) Ethyl](1.0 equivalent, 827 g) and 216 mg of the potassium propyonate (2 mmol) in an emulsion mixture of dichloromethane and water with a 2:1 ratio (20 mL:10 mL) in a 50-mL round-bottom flask, equipped with a stir bar and reflux condenser. The mixture was refluxed for 24 hours, cooled, and the phases were separated using an aqueous workup. The organic phase was evaporated to dryness at reduced pressure to a white powder and was used as is.

The Synthetic Procedure for Bis-(Diphenyltin propyonate) Propane (MA-306): Step 1: [Bis (Triphenylstin) propane]. Triphenyltin Chloride (1.0 equivalent, 5 g) was added to a 2 neck round bottom flask (250 mL) followed a magnetic stir bar. The flask was then sealed with a septa and a glass stopper. The entire setup was then purged with nitrogen for 2 hours to keep the reaction conditions as dry (free of moisture) as possible. Dry tetrahydrofuran (100 mL) was then added through the septa via syringe and stirring was applied for 5 mins to dissolve the solid. High pressure nitrogen was applied through the septa and the glass stopper was removed to add pellets of sodium metal (2.5 equiv, 745 mg) to the flask. The flask was then resealed with the glass stopper and the high-pressure nitrogen was removed from the flask. The mixture was stirred for 24 hours at room temperature. While stirring, propylene-1,3-diol (0.5 equiv, 0.94 ml) was added dropwise. The mixture stirred for an additional 15 minutes. After the reaction ended, the excess sodium was carefully removed and placed on isopropyl alcohol to quench. After the reaction was completed, the mixture was filtered, and the solvent was removed through rotary evaporation. Water (100 mL), ether (100 mL) and benzene (50 mL) were added to dissolve the residue and was placed in a separatory funnel. The combined organic layers were collected, separated, and dried with anhydrous sodium sulfate. The solvent was distilled off under reduced pressure, and white powder was obtained. This compound is stable to air in the solid state but oxidizes slowly in solution.

Step 2: [Bis (Diiphenyltin Iodide) propane]. Bis (Triphenyltin) propane (1.0 equiv, 0.742 mg) was added to a round bottom flask (100 mL) with a magnetic stir bar followed by benzene (70 mL). The flask was then sealed with a septum. While applying stirring at room temperature, a solution of iodine (2.0 equiv, 1.345 g) in methanol (20 mL) was added via syringe, the mixture stirred for 48 hours. After the reaction was completed, the mixture was filtered, and the solvent was removed through rotary evaporation which made a slightly pink powder.

Step 3: [Bis (Diiphenyltin Iodide) propane (1.0 equivalent, 841 g) and 216 mg of the potassium propyonate (2 mmol) in an emulsion mixture of dichloromethane and water with a 2:1 ratio (20 mL:10 mL) in a 50-mL round-bottom flask, equipped with a stir bar and reflux condenser. The mixture was refluxed for 24 hours, cooled, and the phases were separated using an aqueous workup. The organic phase was evaporated to dryness at reduced pressure to a white powder and was used as is.

The Synthetic Procedure for Bis-(Triphenylantimony propyonate) Di-Oxide (MA-311): Step 1: Triphenylantimony Dibromide (2.0 equivalents, 1.024 g) was dissolved in a 25 mL round bottom flask with methanol (15 mL). Lithium peroxide (1.0 equivalent, 45.88 mg) was dissolved in another 25 mL round bottom flask with methanol (15 mL). Both solutions were combined in a 50 mL round bottom flask with a stir bar and reflux condenser and stirred for 2 hours at 70° C. After the reaction was completed, the mixture was filtered, and the solvent was removed through rotary evaporation. The residue was then extracted with DCM (2×30 mL) and placed in a separatory funnel with water. The combined organic layers were dried over Na₂SO₄ and concentrated under vacuum to obtain the desired product as a white solid (Bis-(Triphenylantimony) Di-Oxide).

Step 2: (Bis-(Triphenylantimony) Di-Oxide) (1.0 equivalent, 897 g) and 216 mg of the potassium propyonate (2 mmol) were added to a 50 mL round bottom flask with a stir bar, followed by dichloromethane (20 mL) and water (10 mL). The flask was then connected to a reflux condenser and placed in an oil bath, and the mixture was stirred for 3 hours at 50° C. After the reaction was completed, the mixture was filtered, and the solvent was removed through rotary evaporation. The residue was then extracted with DCM (2×30 mL) and placed in a separatory funnel with water to minimize the presence of potassium bromide. The combined organic layers were dried over Na₂SO₄ and concentrated under vacuum to obtain the desired product as a white solid.

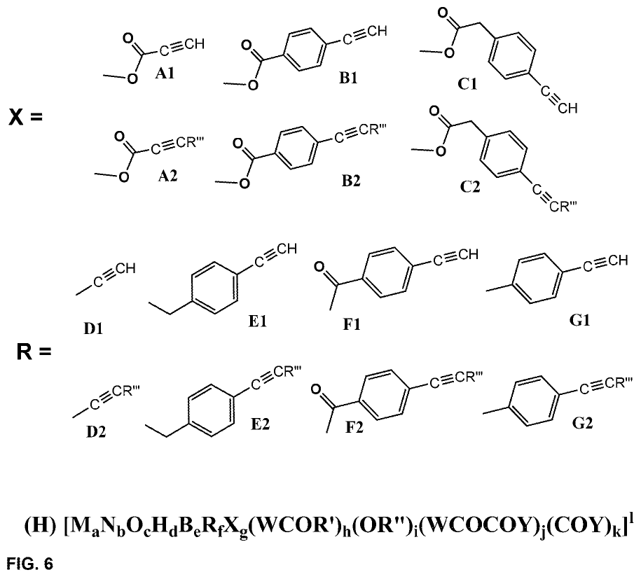

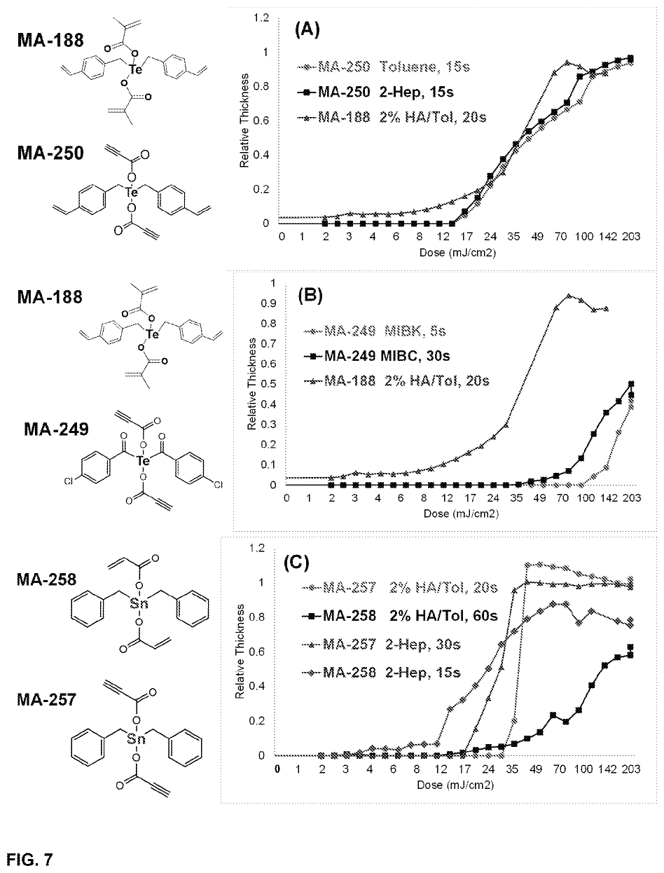

In accordance with embodiments of the present disclosure, an alkyne functional group is used in ligands attached to mononuclear and multinuclear complexes of metal containing resists suitable for use in EUV lithography. Metals are specifically those elements from the main group that strongly absorb EUV light such as: tellurium, antimony, tin, iodine, bismuth and indium. Despite the contention that alkynes do not participate in free-radical reactions, three compounds containing propynoic acid were prepared in the form of the propynoate ligand (FIG. 6A). The plan was to combine these alkyne compounds with azides in an effort to conduct “click” type reactions. However, it was never expected that these alkynyl ligands would have reactivity on their own. Moreover, it was surprisingly found that compounds prepared using the propynoate ligand exhibit EUV reactivity without the use of azides and produce excellent contrast curves.

Overall, embodiments of the present disclosure also include new ligands such as: 4-ethynyl-benzoate, 4-ethynyl-benzyl and ethynyl-benzoyl (FIGS. 6B1, 6C1, 6E1, respectively). These ligands can be incorporated into the mononuclear compounds listed above, and also into multinuclear complexes of the type shown in FIG. 6H, where ligands A1, A2, B1, B2, C1, C2 take the form of WCOR′, ligands D1, D2, E1, E2, G1, G2 takes the form of R and ligands F1 and F2 takes the form of WCOR′ where W is absent, and R′″=—CH₃, —CH₂CH₃, or —C₆H₅.

Unexpected Results: During experiments conducted at Paul Shirrer Institute Nov. 22-28, 2021, resists MA-188, MA-250, MA-249, MA-258 and MA-257 were exposed to EUV light using an open-frame exposure pattern over a range of doses. The first three compounds are mononuclear tellurium compounds whereas the last two are mononuclear tin compounds. After development in selected solvents and measurement of film thickness vs. dose, contrast curves were created (FIGS. 7A-7C). FIG. 7A shows that MA-250 with two propynoate ligands creates a contrast curve with a cleaner toe vs. MA-188 that contains methacrylate ligands. FIG. 7B also shows that MA-249 with two propynoate ligands creates a contrast curve with a cleaner toe vs. MA-188 that contains methacrylate ligands. FIG. 7C makes the best “apples-to-apples” comparison between MA-257 with two propynoate ligands vs. MA-258 with two acrylate ligands. The propynoate ligands clearly provide cleaner toes and higher contrast than the acrylate ligands, using similar development conditions. These latter two compounds are mononuclear tin compounds, so there may be something particularly beneficial when combining the propynoate ligands with tin.

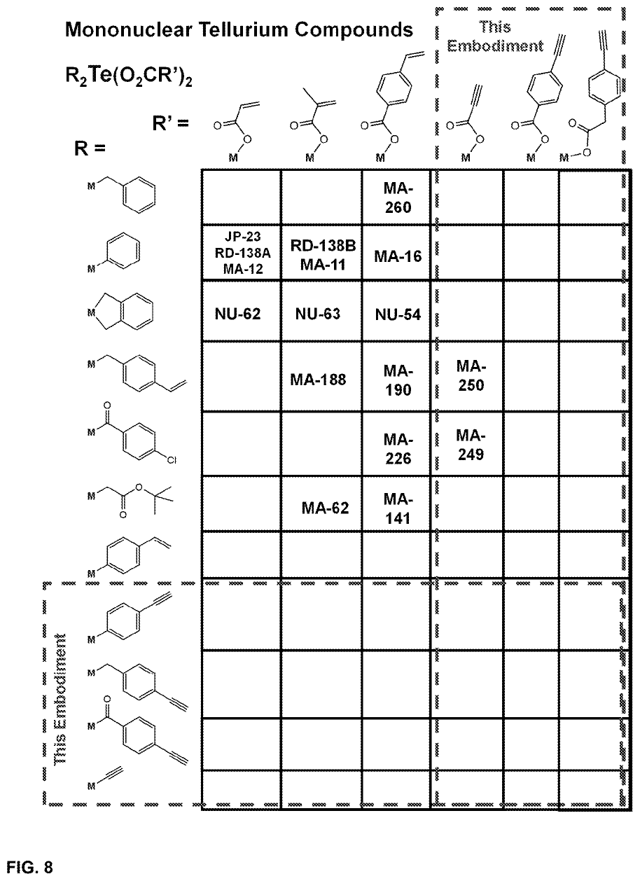

Mononuclear Target Molecules: FIGS. 8-11 compares previously made mononuclear complexes of tellurium, tin, antimony and bismuth, respectively. In embodiments, the compound Ph₂Te(O₂CCH═CH₂)₂ has three names: JP-23, RD-138A, MA-12, respectively. These four figures (FIGS. 8-11) also outline specific compound types (shown as “This Embodiment”) that can be made in the future to further elucidate the benefits and opportunities of using the ligands containing alkynes (FIG. 6) in mononuclear complexes.

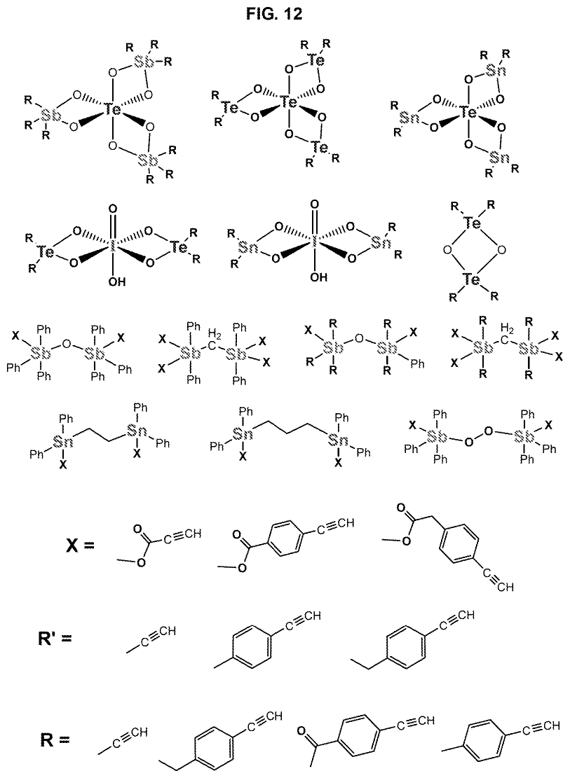

Multinuclear Complexes: FIG. 12 shows multinuclear complexes that can be made including ligands containing alkynes (FIG. 6) based on previously synthesized multinuclear complexes using analogous ligands. For the design of clusters, it is important to distinguish between R and X groups, where R groups have carbons bonded to the metals and X groups where carboxylates are bonded to the metals. To date, most of the outer ligands in the clusters we have explored have been R groups.

Referring now to FIG. 7, FIG. 7 depicts contrast curves comparing mononuclear complexes containing alkynes and alkenes. In all cases, the complexes containing alkyne ligands show superior contrast curves (vs. alkene ligands) with either cleaner toes, higher contrast or both.

Referring now to FIG. 8, FIG. 8 depicts mononuclear complexes of tellurium that have been evaluated using EUV lithography by our group shown in bold with the alpha numeric labels. The areas marked “This Embodiment” define compounds using the new alkynyl ligands that we will synthesize and evaluate lithographically.

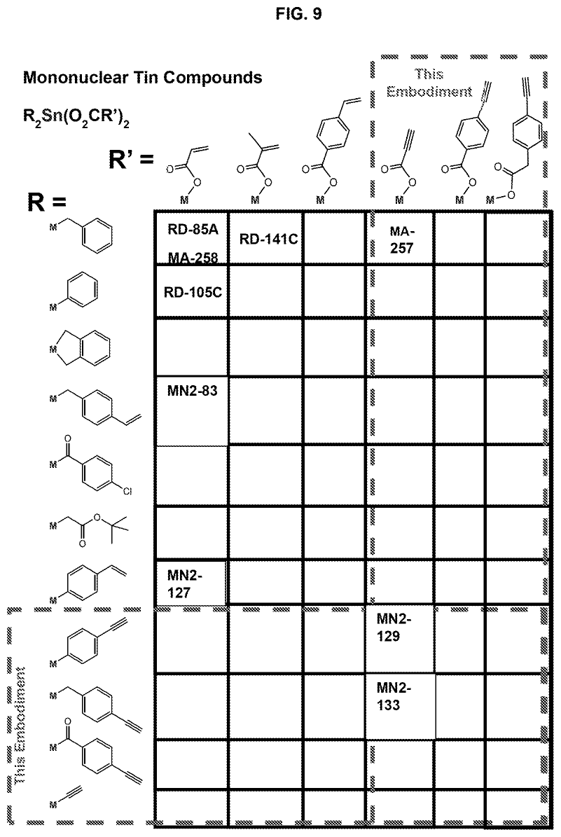

Referring now to FIG. 9, FIG. 9 depicts mononuclear complexes of tin that have been evaluated using EUV lithography by our group shown in bold with the alpha numeric labels. The areas marked “This Embodiment” define compounds using the new alkynyl ligands that we will synthesize and evaluate lithographically.

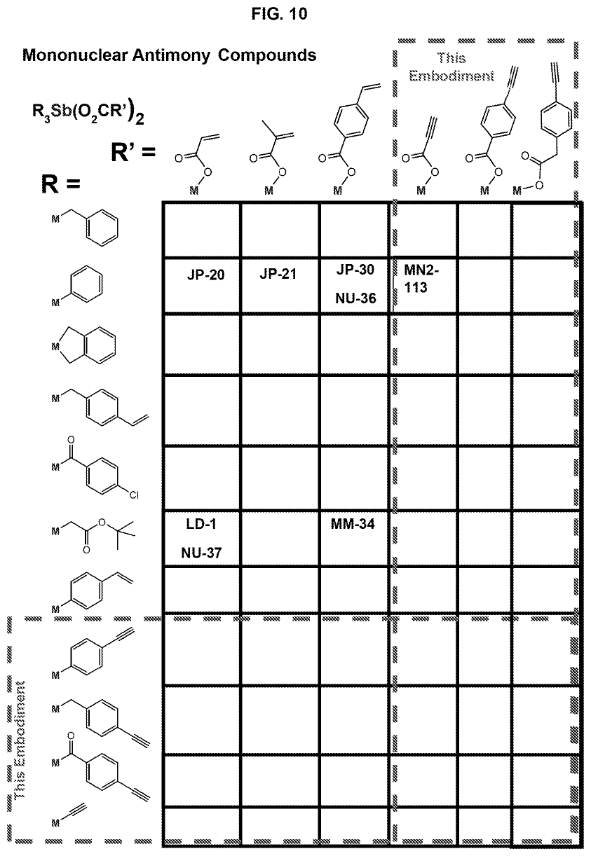

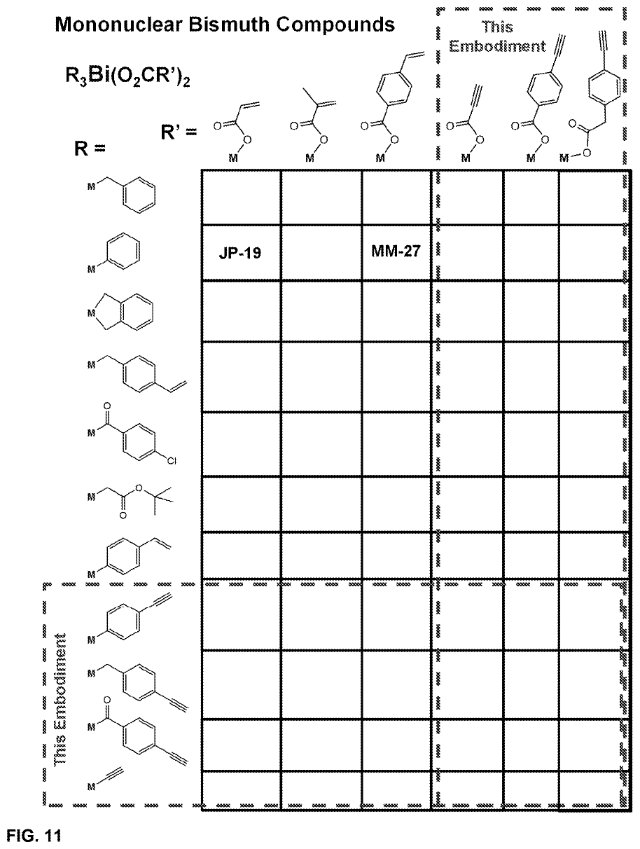

Referring now to FIG. 10, FIG. 10 depicts mononuclear complexes of antimony that have been evaluated using EUV lithography by our group shown in bold with the alpha numeric labels. The areas marked “This Embodiment” define compounds using the new alkynyl ligands that we will synthesize and evaluate lithographically.

Referring now to FIG. 11, FIG. 11 depicts mononuclear complexes of bismuth that have been evaluated using EUV lithography by our group shown in bold with the alpha numeric labels. The areas marked “This Embodiment” define compounds using the new alkynyl ligands that we will synthesize and evaluate lithographically.

Referring now to FIG. 12, FIG. 12 depicts multinuclear target compounds that could be made to evaluate the benefits of alkyne ligands as EUV Photoresists.

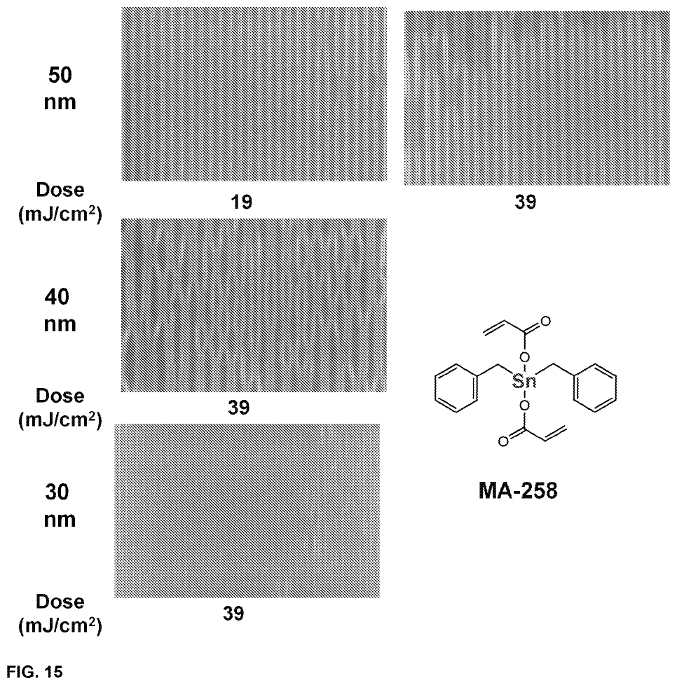

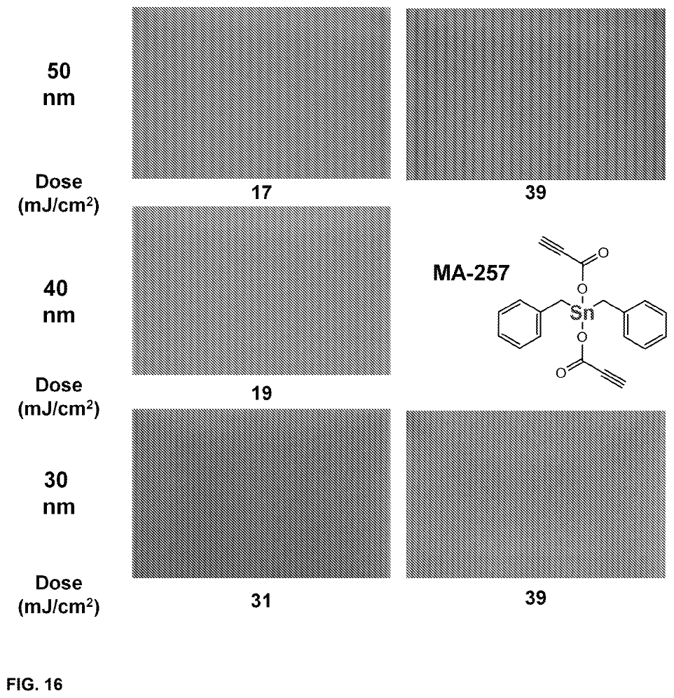

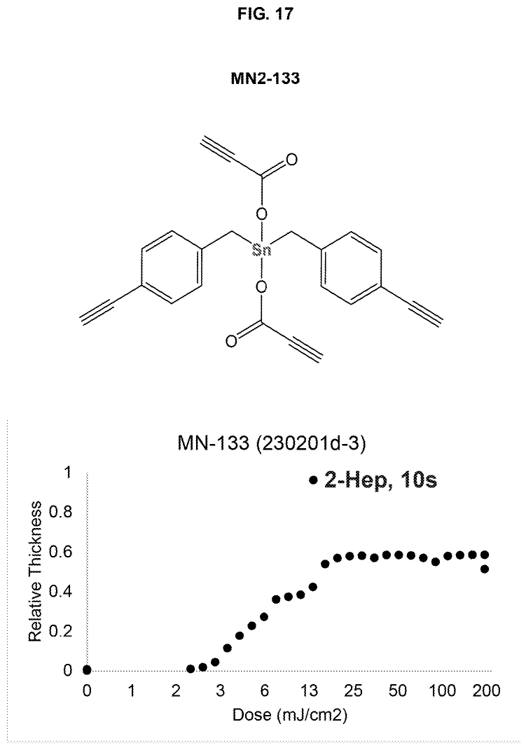

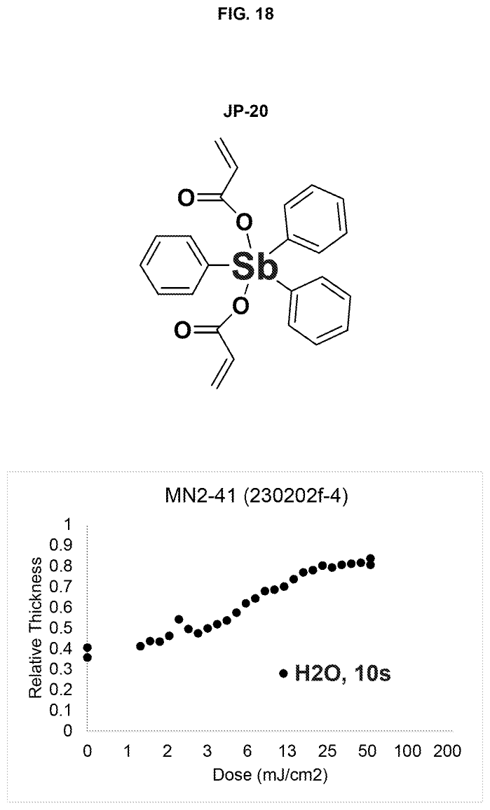

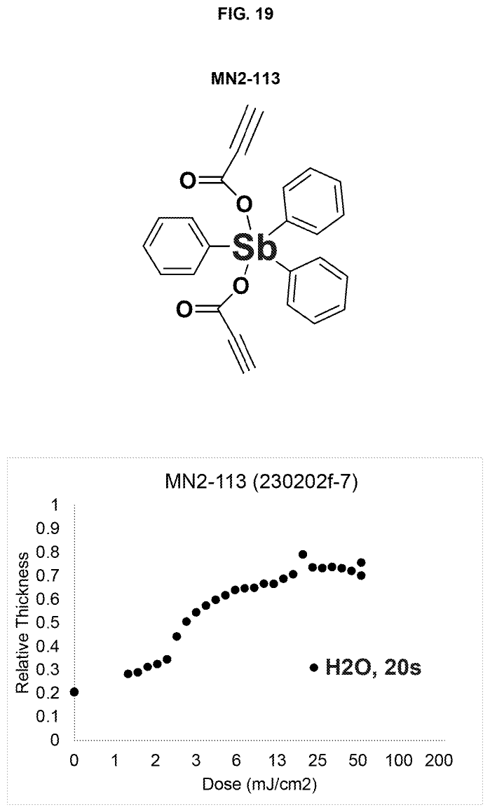

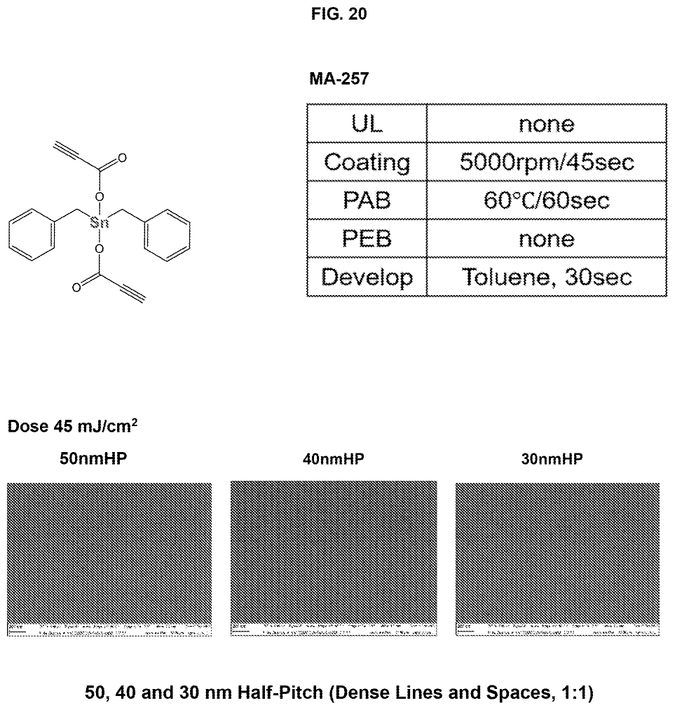

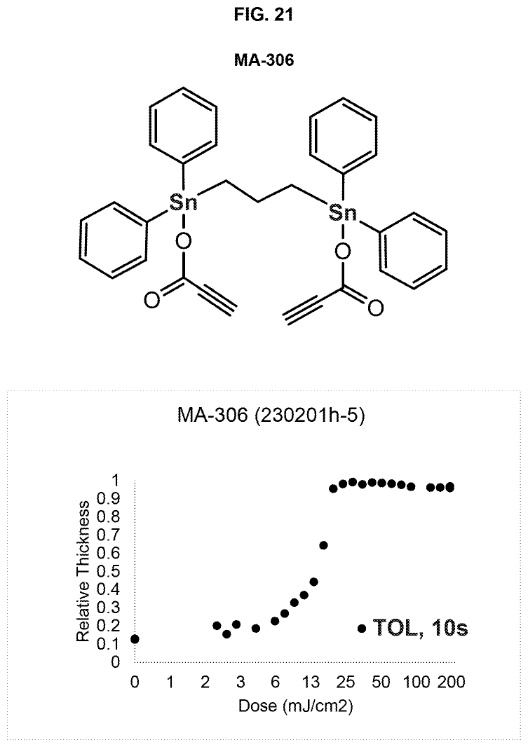

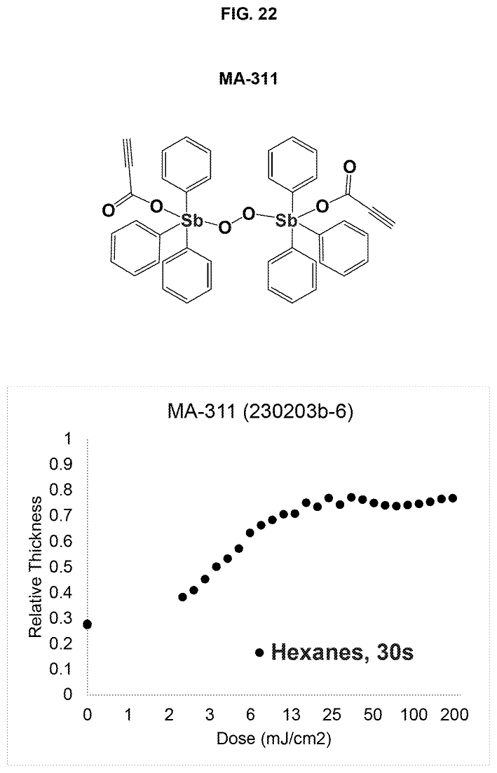

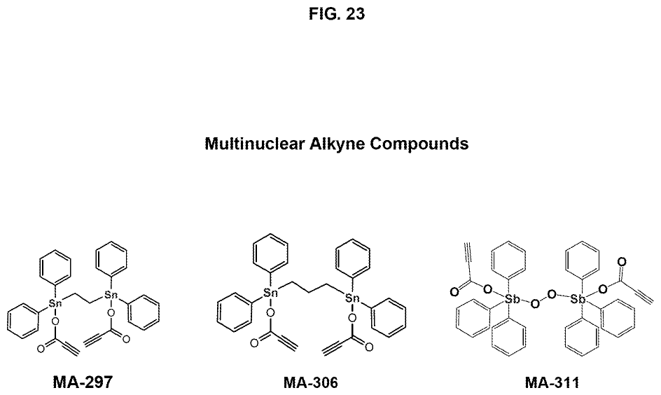

Preliminary imaging results are shown in FIGS. 15 and 16 for resists MA-258 and MA-257. Both resists were imaged using interference lithography and a 30-second development in 2-heptanone. These imaging results are the first for these compounds and are completely unoptimized. FIG. 15 shows the olefin resist, MA-258, prints 50-nm dense lines with some line collapse problems. The 40-nm lines show lots of line collapse problems, and the 30-nm lines are not fully resolved. On the other hand, FIG. 16 shows that the alkynyl resist, MA-257, prints excellent dense lines with resolution of 50-, 40- and 30-nm resolution. Clearly, MA-257 is showing better imaging capabilities than dose MA-258.

The entire disclosure of all applications, patents, and publications cited herein are herein incorporate by reference in their entirety. While the foregoing is directed to embodiments of the present disclosure, other and further embodiments of the disclosure may be devised without departing from the basic scope thereof.

The present invention is not to be limited by the above description, but to be defined by the appended claims and their equivalents.

What is claimed is:

1. A mononuclear lithographic composition, represented by the below formula,
RₐM(O₂CR′)₂,
wherein in M is tellurium (Te), antimony (Sb), tin (Sn), or bismuth (Bi), a is 2-3, and at least one of R and R′, include an alkyne functional group moiety.

2. A mononuclear lithographic composition of claim 1, wherein M is Te, and a=2.

3. A mononuclear lithographic composition of claim 1, wherein M is Sn, and a=2.

4. A mononuclear lithographic composition of claim 1, wherein M is Sb, and a=3.

5. A mononuclear lithographic composition of claim 1, wherein M is Bi, and a=3.

6. The mononuclear lithographic composition of claim 1, wherein
R, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, -o-C₆H₄OCH₃, -m-C₆H₄OCH₃, -p-C₆H₄OCH₃, —C₆H₄CH₂CH₃, -o-CH₂C₆H₄OCH₃, -m-CH₂C₆H₄OCH₃, -p-CH₂C₆H₄OCH₃, —C₆H₁₁, —C≡CH, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH, —C≡CR′″, —CH₂C₆H₄C≡CR′″, —(CO)C₆H₄C≡CR′″, or —C₆H₄C≡CR′″,
R′, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CH(OCH₃)(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, -o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH, —C≡CR′″, —C₆H₄C≡CR′″, or —CH₂C₆H₄C≡CR′″;
R′″, is independently an aromatic or aliphatic hydrocarbon selected from: —CH₃, —CH₂CH₃, or —C₆H₅;
O is each independently oxygen; and
C is each independently carbon.

7. A multinuclear lithographic composition, represented by the below formula,
[MₐN_{b}O_{c}H_{d}BₑR_{f}X_{g}(WCOR′)ₕ(OR″)ᵢ(WCOCOY)ⱼ(COY)ₖ]l,
wherein the composition contains at least one of R, R′ and R″, and at least one of R, R′ and R″ includes an alkyne functional group moiety:
R, when present, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —C≡CH, —CH₂C₆H₄C≡CH, —(CO)C₆H₄C≡CH, —C≡CR′″, —CH₂C₆H₄C≡CR′″, —(CO)C₆H₄C≡CR′″, or —C₆H₄C≡CR′″-o-C₆H₄OCH₃, -m-C₆H₄OCH₃, -o-CH₂C₆H₄OCH₃, -m-CH₂C₆H₄OCH₃, -p-CH₂C₆H₄OCH₃, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH;
R′, when present, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CH(OCH₃)(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, —C₆H₄C≡CH, —CH₂C₆H₄C≡CH, —C≡CR′″, —C₆H₄C≡CR′″, or —CH₂C₆H₄C≡CR′″, -o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH;
R″, when present, is each independently an element, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —C₆H₄C≡CH, —CH₂C₆H₄C≡CH, —C₆H₄C≡CR′″, or —CH₂C₆H₄C≡CR′″;
R′″, when present, is independently an aromatic or aliphatic hydrocarbon selected from: —CH₃, —CH₂CH₃, or —C₆H₅;
M is each independently an element selected from the group of tellurium (Te), antimony (Sb), tin (Sn), iodine (I), bismuth (Bi) and indium (In);
N, when present, is each independently an element selected from the group consisting of indium (In), tin (Sn), antimony (Sb), tellurium (Te), bismuth (Bi) and iodine (I);
H, when present, is each independently hydrogen;
O is each independently oxygen;
B, when present, is each independently selected from W, C₂O₄, SO₄, PO₄, (CH₂)₂C₆H₂(CH₂)₂, —C≡C—, —CH₂C≡C—, —CH₂C≡CCH₂—, or —CH₂C₆H₄CH₂—, —O—O—, —O₂CCH₂CH₂CO₂—, —O₂CCH═CHCO₂—, —O₂CC≡CCO₂—, —(OCO)₂NCH₂CH₂N(COO)₂—,
X, when present, is each independently selected from F, Cl, Br, CN, I or C₂O₄, an alkyne functional group moiety, —O₂CC≡CH, —O₂CC₆H₄C≡CH, or —O₂CCH₂C₆H₄C≡CH;
W, when present, is each independently an element or a compound selected from the group consisting of —CH₂, NR′, S, and O;
Y when present, is each independently an element or compound selected from the group consisting of —WR′, and —R′, wherein
a=1-8; b=0-5; c=0-20; d=0-20; e=0-10; f=0-20; g=0-20; h=0-5; i=0-5; j=0-5; k=0-6; and I=charge on an ion or a complex selected from: −4, −3, −2, −1, 0, +1, +2, +3, +4.

8. A multinuclear lithographic composition, of claim 7, wherein

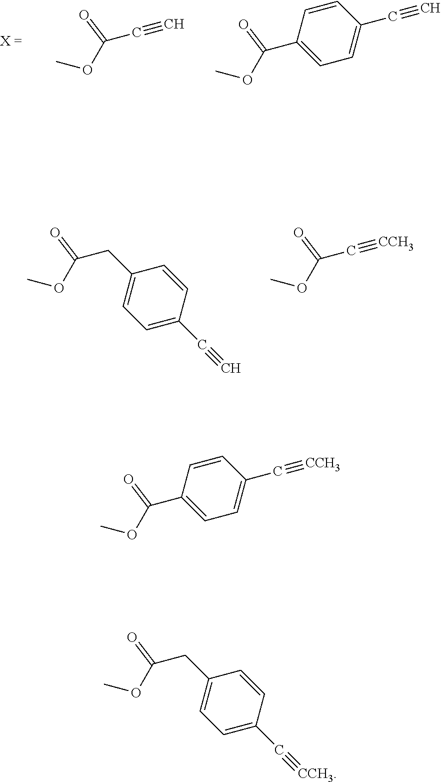

9. A multinuclear lithographic composition, of claim 7, wherein

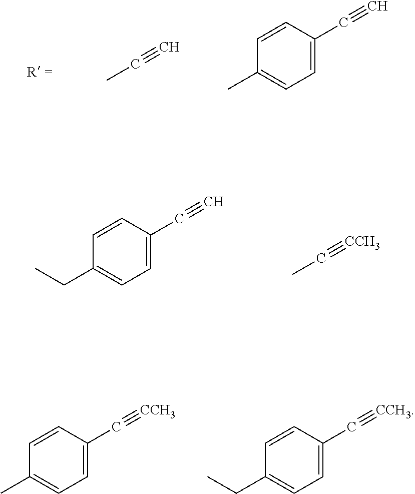

10. A multinuclear lithographic composition, of claim 7, wherein

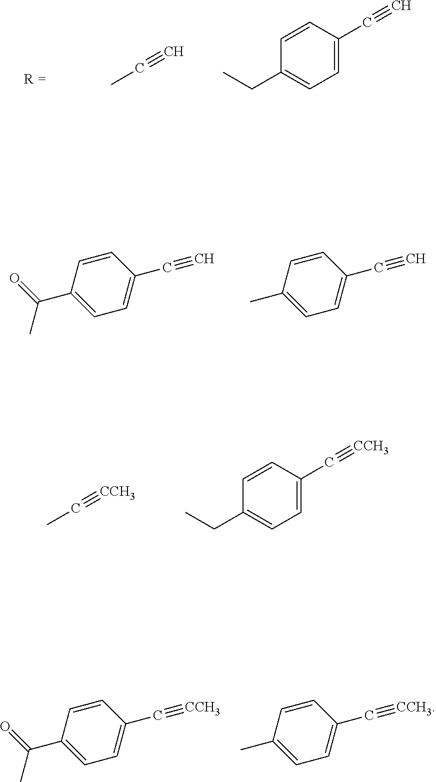

11. A coating solution comprising: an organic solvent, and an organometallic composition represented by the formula
RₐM(O₂CR′)₂,
wherein in M is tellurium (Te), antimony (Sb), tin (Sn), or bismuth (Bi), a is 2-3, and at least one of R and R′, include an alkyne functional group moiety.

12. The coating solution of claim 11, wherein M is Te, and a=2.

13. The coating solution of claim 11, wherein M is Sn, and a=2.

14. The coating solution position of claim 11, wherein M is Sb, and a=3.

15. The coating solution of claim 11, wherein M is Bi, and a=3.

16. The coating solution of claim 11, wherein
R, is independently an aromatic or aliphatic hydrocarbon selected from: —C₆H₅, —CH₃, —CH₂CH₃, —CH(CH₃)₂, —C(CH₃)₃, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂C₆H₄CH═CH₂, —C₆H₄OCH₃, p-C₆H₄OCH₃, —C₆H₄CH₂CH₃, —CH₂C₆H₄OCH₃, —C₆H₁₁, —C≡CH, —(CO)C₆H₄C≡CH, —C≡CR′″, —CH₂C₆H₄C≡CR′″, —(CO)C₆H₄C≡CR′″, or —C₆H₄C≡CR′″-o-C₆H₄OCH₃, -m-C₆H₄OCH₃, -o-CH₂C₆H₄OCH₃, -m-CH₂C₆H₄OCH₃, -p-CH₂C₆H₄OCH₃, -o-CH₂C₆H₄C≡CH, -m-CH₂C₆H₄C≡CH, -p-CH₂C₆H₄C≡CH, —(CO)C₆H₄C≡CH, -o-C₆H₄C≡CH, -m-C₆H₄C≡CH, -p-C₆H₄C≡CH;
R′, is independently an element comprising oxygen, nitrogen, fluorine, chlorine, bromine, or iodine, an aromatic hydrocarbon, or an aliphatic hydrocarbon selected from: —H, —CH₃, —CH₂CH₃, —CH(CH₃)₂, —C(CH₃)₃, —C₆H₅, —CH═CH₂, —C(CH₃)═CH₂, —CH₂CH═CH₂, —CH₂C≡CH, —CH₂C≡N, —CH₂C₆H₅, —C₆H₄CH═CH₂, —C₆H₄C(CH₃)═CH₂, —CH₂CH₂F, —CH₂CH₂Cl, —CH₂CH₂Br, —CH₂CH₂I, —CH₂CH₂OCH₃, —CH₂CH₂CN, —CHF(CH₃), —CHCl(CH₃), —CHBr(CH₃), —CHI(CH₃), —CHOCH₃(CH₃), —CHCN(CH₃), —CH₂F, —CH₂Cl, —CH₂Br, —CH₂I, —CH₂OCH₃, —CH₂CN, —OCH₃, —OCH₂CH₃, —OC(CH₃)₃, —C₆H₄OCH₃, —C₆H₄Cl, —C≡CH, —C₆H₄C≡CH, —CH₂C₆H₄C≡CH, —C≡CR′″, —C₆H₄C≡CR′″, or —CH₂C₆H₄C≡CR″ -o-C₆H₄C≡CH, -m-C₆H₄C≡CH, -p-C₆H₄C≡CH, -o-CH₂C₆H₄C≡CH, -m-CH₂C₆H₄C≡CH, -p-CH₂C₆H₄C≡CH;
R′″, is independently an aromatic or aliphatic hydrocarbon selected from: —CH₃, —CH₂CH₃, or —C₆H₅;
O is each independently oxygen; and
C is each independently carbon.

17. The coating solution of claim 11, wherein the solvent is an alcohol, an ester, or a mixture thereof.

18. A method for forming a radiation patternable coating, the method comprising: contacting a coating solution of claim 11, with a substrate under conditions suitable for forming a film atop the substrate.

19. The method of claim 18, wherein the substrate is heated to a temperature from about 30 degrees Celsius to about 250 degrees Celsius for about 0.5 minutes to about 30 minutes.

20. The method of claim 18, wherein the coating solution is spin coated to form a film atop the substrate.
